SEQUENCE LISTING (8 sequences)

SEQ ID NO: 1 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 2 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 3 - protein, 5 aa, Homo sapiens
SYTMH

SEQ ID NO: 4 - protein, 17 aa, Homo Sapiens
FISYDGNNKYYADSVKG

SEQ ID NO: 5 - protein, 9 aa, Homo sapiens
TGWLGPFDY

SEQ ID NO: 6 - protein, 12 aa, Homo sapiens
RASQSVGSSYLA

SEQ ID NO: 7 - protein, 7 aa, Homo sapiens
GAFSRAT

SEQ ID NO: 8 - protein, 9 aa, Homo sapiens
QQYGSSPWT

(19) United States
(12) Patent Application Publication
(10) Pub. No.: US 2015/0079100 A1
(43) Pub. Date: Mar. 19, 2015

(54) METHODS OF TREATMENTS USING CTLA-4 ANTIBODIES

(71) Applicant: BRISTOL-MYERS SQUIBB COMPANY, Princeton, NJ (US)

(72) Inventors: Amit Roy, Woodbury, CT (US); Yan Feng, Pittsburgh, PA (US); Bruce C. Stouffer, Branchburg, NJ (US)

(21) Appl. No.: 14/386,917

(22) PCT Filed: Mar. 22, 2013
(86) PCT No.: PCT/US2013/033513

§ 371 Date: Sep. 22, 2014

Related U.S. Application Data
(60) Provisional application No. 61/614,854, filed on Mar. 23, 2012.
(60) Provisional application No. 61/791,325, filed on Mar. 15, 2013.

(51) Int. Cl. C07K 16/28; A61K 39/395; G01N 33/574; C07K 16/30

(52) U.S. Cl. CPC C07K 16/2818; C07K 16/3053; A61K 39/39558; G01N 33/57492; C07K 2317/21; C07K 2317/565; A61K 2039/545
USPC 424/142.1; 435/7.24

(57) ABSTRACT

In certain embodiments, the present invention, the present invention provides a method of treating a cancer in a subject, comprising: (a) administering to the subject a predetermined dosage of an anti-CTLA4 antibody; (b) detecting the level of the anti-CTLA4 antibody in a sample of the subject; and (c) increasing the dosage of the anti-CTLA4 antibody in the subject if the level of the anti-CTLA4 antibody from step (b) is below a threshold exposure level, such that the cancer is treated.

### CROSS-REFERENCE TO RELATED APPLICATIONS

This application claims priority of U.S. Provisional Application Ser. No. 61/614,854, filed on Mar. 23, 2012, and U.S. Provisional Application Ser. No. 61/791,325, filed on Mar. 15, 2013, each of which is herein incorporated by reference.

### BACKGROUND OF THE INVENTION

T cell immune response is a complex process that involves cell-cell interactions, particularly between T and accessory cells such as APC's, and production of soluble immune mediators (cytokines or lymphokines). This response is regulated by several T-cell surface receptors, including the T-cell receptor complex and other “accessory” surface molecules. Many of these accessory molecules are naturally occurring cell surface differentiation (CD) antigens defined by the reactivity of monoclonal antibodies on the surface of cells.

CD28 antigen, a homodimeric glycoprotein of the immunoglobulin superfamily, is an accessory molecule found on most mature human T cells. Current evidence suggests that this molecule functions in an alternative T cell activation pathway distinct from that initiated by the T-cell receptor complex. Monoclonal antibodies (MAbs) reactive with CD28 antigen can augment T cell responses initiated by various polyclonal stimuli. These stimulatory effects may result from MAb-induced cytokine production as a consequence of increased mRNA stabilization.

CTLA4 (cytotoxic T lymphocycte-associated antigen-4) is accepted as opposing CD28 activity and dampening T cell activation. CTLA4 deficient mice suffer from massive lymphoproliferation. It has been reported that CTLA4 blockade augments T cell responses in vitro and in vivo, exacerbates antitumor immunity, and enhances an induced autoimmune disease. It has also been reported that CTLA4 has an alternative or additional impact on the initial character of the T cell immune response. This is consistent with the observation that some autoimmune patients have autoantibodies to CTLA4. It is possible that CTLA4 blocking autoantibodies play a pathogenic role in these patients. Non-human CTLA4 antibodies have been used in the various studies discussed above. Furthermore, human antibodies against human CTLA4 have been described as immunostimulation modulators in a number of disease conditions, such as treating or preventing viral and bacterial infection and for treating cancer.

There continues to be a need for methods of administering an optimum dose of a CTLA4 antibody for the treatment of a disease, such as cancer or infectious disease, to a patient, that results in a partial or complete response, and minimizes the incidence and/or severity of an adverse event.

### SUMMARY OF THE INVENTION

In certain embodiments, the present invention provides a method of treating a CTLA4-related disease (e.g., a cancer) in a subject in need of treatment. Such method comprises: (a) administering to the subject a predetermined dosage of an anti-CTLA4 antibody (e.g., 3 mg/kg or 10 mg/kg of body weight); (b) detecting the level of the anti-CTLA4 antibody in a sample of the subject; and (c) increasing the dosage of the anti-CTLA4 antibody in the subject if the level of the anti-CTLA4 antibody from step (b) is below a threshold exposure level, such that the CTLA4-related disease (e.g., cancer) in the subject is treated. In certain embodiments, step (c) of the method comprises not increasing the dosage of the anti-CTLA4 antibody in the subject if the level of the anti-CTLA4 antibody from step (b) is at or above a threshold exposure level.

Optionally, the anti-CTLA4 antibody used in the methods is a human antibody. Preferably, the anti-CTLA4 antibody is MDX-010 (also referred to as ipilimumab or Yervoy). An exemplary anti-CTLA4 antibody comprises: (a) a heavy chain variable region comprising the amino acid sequence of SEQ ID NO: 17; and (b) a light chain variable region comprising the amino acid sequence of SEQ ID NO: 7. Another exemplary anti-CTLA4 antibody comprises: (a) a heavy chain variable region CDR1 comprising SEQ ID NO: 27; (b) a heavy chain variable region CDR2 comprising SEQ ID NO: 32; (c) a heavy chain variable region CDR3 comprising SEQ ID NO: 37; (d) a light chain variable region CDR1 comprising SEQ ID NO: 24; (e) a light chain variable region CDR2 comprising SEQ ID NO: 29; and (f) a light chain variable region CDR3 comprising SEQ ID NO: 35.

In certain aspects, step (b) of the above described method is performed by detecting the level of the anti-CTLA4 antibody via an immunoassay. For example, the immunoassay comprises contacting said sample with an antigen which binds to the anti-CTLA4 antibody under conditions suitable for antibody-antigen complex formation, followed by the detection of the antibody-antigen complex formation. Preferably, the antigen which binds to the anti-CTLA4 antibody is a CTLA4 protein (e.g., a CTLA4/Fc fusion protein). Optionally, detection is accomplished by a means selected from the group consisting of EIA, ELISA, RIA, indirect competitive immunoassay, direct competitive immunoassay, non-competitive immunoassay, sandwich immunoassay, and agglutination assay.

In certain aspects, the CTLA4-related disease of the above-described method is a cancer. Examples of the cancer include, but are not limited to, melanoma, prostate cancer, lung cancer, gastric cancer, ovarian cancer, breast cancer, and glioblastoma. In other aspects, the CTLA4-related disease of the above-described method is an infectious disease.

In certain embodiments, the present invention provides a method of decreasing clearance of a therapeutic anti-CTLA4 antibody in a subject in need of treatment, comprising: (a) administering to the subject a predetermined dosage of an anti-CTLA4 antibody (e.g., 3 mg/kg or 10 mg/kg of body weight); (b) detecting the level of the anti-CTLA4 antibody in a sample of the subject; and (c) increasing the dosage of the anti-CTLA4 antibody in the subject if the level of the anti-CTLA4 antibody from step (b) is below a threshold exposure level, such that clearance of the anti-CTLA4 antibody is decreased in the subject. Preferably, the subject is treated for a CTLA4-related disease (e.g., a cancer). Examples of the cancer include, but are not limited to, melanoma, prostate cancer, lung cancer, gastric cancer, ovarian cancer, breast cancer, and glioblastoma.

In certain embodiments, the present invention provides a kit comprising: (1) an antigen which specifically binds to an anti-CTLA4 antibody; and (2) reagents necessary for facilitating an antibody-antigen complex formation. Optionally, the kit further comprises an anti-CTLA4 antibody (e.g., MDX-010) as a control. To illustrate, the antigen which specifically binds to an anti-CTLA4 antibody is a CTLA4 protein (e.g., a CTLA4/Fc fusion protein).

### BRIEF DESCRIPTION OF THE DRAWINGS

FIG. 1A shows predicted ipilimumab Cminss values as a function of time during induction with the 3 mg/kg dose.

FIG. 1B shows predicted ipilimumab Cminss values as a function of time during induction with the 10 mg/kg dose.

FIG. 2A shows boxplots of the distributions of Cminss by ipilimumab dose relative to target trough concentration.

FIG. 2B shows boxplots of the distributions of Cavgss by ipilimumab dose relative to target trough concentration.

FIG. 2C shows boxplots of the distributions of Cminss of ipilimumab by dose and population (previously untreated and treated) with targets.

FIG. 2D shows boxplots of the distribution of ipilimumab Cminss following the first dose of the drug by dose and population (previously untreated and treated), relative to target trough concentrations.

FIG. 2E shows boxplots of the distribution of ipilimumab Cminss following the third dose of the drug by dose and population (previously untreated and treated), relative to target trough concentrations.

FIG. 2F shows boxplots of the distribution of ipilimumab Cavgss by dose and population (previously untreated and treated), relative to target trough concentrations.

FIG. 3 shows estimated relative hazard ratios of covariates in final CPH model.

FIG. 4A shows the amino acid sequence (SEQ ID NO: 7) of the light chain variable region of the 10D1 human monoclonal antibody (also referred to as ipilimumab). The CDR1 (SEQ ID NO: 24), CDR2 (SEQ ID NO: 29) and CDR3 (SEQ ID NO: 35) regions are delineated.

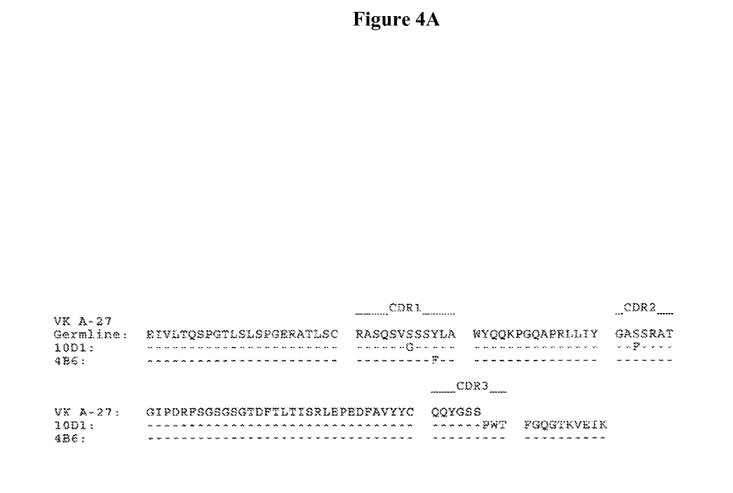

FIG. 4B shows the amino acid sequence (SEQ ID NO: 17) of the heavy chain variable region of the 10D1 human monoclonal antibody (also referred to as ipilimumab). The CDR1 (SEQ ID NO: 27), CDR2 (SEQ ID NO: 32) and CDR3 (SEQ ID NO: 37) regions are delineated.

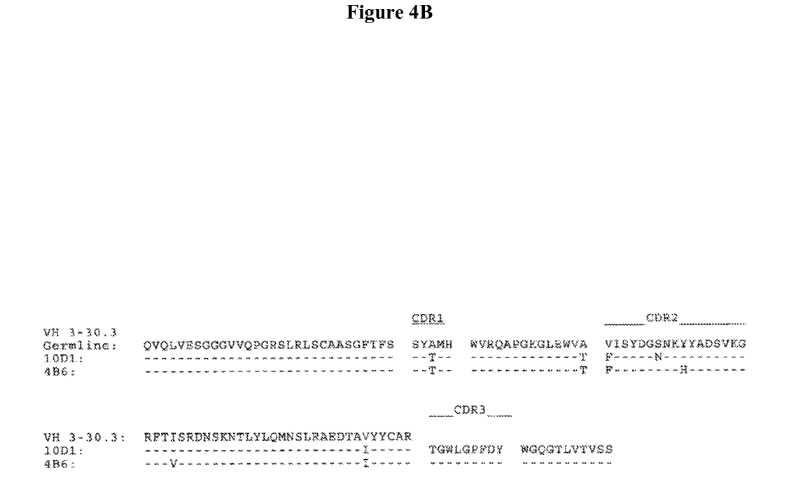

### DETAILED DESCRIPTION OF THE INVENTION

In certain embodiments, the present invention relates to therapeutic methods using isolated monoclonal antibodies, particularly human monoclonal antibodies, which bind specifically to CTLA4 (herein referred to as “CTLA4 antibodies” or “anti-CTLA4 antibodies”).

In a specific embodiment, the invention provides a method of treating a CTLA4-related disease (e.g., a cancer), which comprise: (a) administering to the subject a predetermined dosage of an anti-CTLA4 antibody; (b) detecting the level of the anti-CTLA4 antibody in a sample of the subject; and (c) increasing the dosage of the anti-CTLA4 antibody to the subject if the level of the anti-CTLA4 antibody from step (b) is below a threshold exposure level, such that the CTLA4-related disease in the subject is treated. In certain embodiments, step (c) of the method comprises not increasing the dosage of the anti-CTLA4 antibody in the subject if the level of the anti-CTLA4 antibody from step (b) is at or above a threshold exposure level. For example, an anti-CTLA4 antibody is administered to the subject at a predetermined dosage of 3 mg/kg or 10 mg/kg of body weight. In certain aspects, the CTLA4-related disease is a cancer. Examples of the cancer include, but are not limited to, melanoma, prostate cancer, lung cancer, gastric cancer, ovarian cancer, breast cancer, and glioblastoma. To illustrate, the subject may be previously treated or untreated for the cancer, before receiving the administration of an anti-CTLA4 antibody.

In another specific embodiment, the present invention provides a method of decreasing clearance of a therapeutic anti-CTLA4 antibody in a subject in need of treatment, comprising: (a) administering to the subject a predetermined dosage of an anti-CTLA4 antibody; (b) detecting the level of the anti-CTLA4 antibody in a sample of the subject; and (c) increasing the dosage of the anti-CTLA4 antibody in the subject if the level of the anti-CTLA4 antibody from step (b) is below a threshold exposure level, such that clearance of the anti-CTLA4 antibody is decreased in the subject. For example, an anti-CTLA4 antibody is administered to the subject at a predetermined dosage of 3 mg/kg or 10 mg/kg of body weight. Preferably, the subject is treated for a CTLA4-related disease (e.g., a cancer). Examples of the cancer include, but are not limited to, melanoma, prostate cancer, lung cancer, gastric cancer, ovarian cancer, breast cancer, and glioblastoma. To illustrate, the subject may be previously treated or untreated for the cancer, before receiving the administration of an anti-CTLA4 antibody.

In another specific embodiment, the present invention provides methods of detecting an anti-CTLA4 antibody in a biologic sample (e.g., a blood sample such as serum or plasma).

In another specific embodiment, the present invention provides a kit comprising: (1) an antigen which specifically binds to an anti-CTLA4 antibody; and (2) reagents necessary for facilitating an antibody-antigen complex formation. Optionally, the kit further comprises an anti-CTLA4 antibody (e.g., MDX-010) as a control. To illustrate, the antigen which specifically binds to an anti-CTLA4 antibody is a CTLA4 protein (e.g., a CTLA4/Fc fusion protein).

### GENERAL DEFINITIONS

In order that the present invention may be more readily understood, certain terms are first defined. Additional definitions are set forth throughout the detailed description.

The term “CTLA4 protein” as used herein, includes a full-length CTLA4 protein, CTLA4 protein fragments, CTLA4 protein variants, and CTLA4 fusion proteins (e.g., CTLA4/Fc fusion protein), which an anti-CTLA4 antibody (e.g., MDX-010) can bind.

The terms “patient” or “subject” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

The term “treating” includes the administration of CTLA4 antibodies of the present invention to prevent or delay the onset of the symptoms, complications, or biochemical indicia of a disease, alleviating the symptoms or arresting or inhibiting further development of the disease, condition, or disorder (e.g., cancer, an infectious disease, or an autoimmune disease). Treatment may be prophylactic (to prevent or delay the onset of the disease, or to prevent the manifestation of clinical or subclinical symptoms thereof) or therapeutic suppression or alleviation of symptoms after the manifestation of the disease.

The term “dosage” or “dose” as used herein, refers to an amount of an anti-CTLA4 antibody which is administered to a subject.

The term “therapeutically effective dosage,” as used herein, refers to a dosage of an anti-CTLA4 antibody which preferably results in a decrease in severity of disease symptoms, an increase in frequency and duration of disease symptom-free periods, an increase in overall survival, or a prevention of impairment or disability due to the disease affliction. One of ordinary skill in the art would be able to determine such amounts based on such factors as the subject's size, the severity of the subject's symptoms, and the particular composition or route of administration selected.

The term “threshold exposure level”, as used herein, refers to a minimum exposure level which allows for clinically meaningful induction and/or maintenance of disease remission (e.g., an increase in overall survival) after administering an anti-CTLA4 antibody in a subject during the induction phase and/or maintenance phase. The threshold exposure level can be readily determined, such as by the exposure-response analyses as described in the working examples. For example, the threshold exposure level can be a trough concentration ranging from 5-120 μg/mL (e.g., 5, 10, 15, 20, 25, 30, 35, 40, 45, 50, 55, 60, 65, 70, 75, 80, 85, 90, 95, 100, 105, 110, 115 or 120 μg/mL). As shown in the working examples, the threshold exposure level may increase, if the dose increases. To illustrate, if the dose is 3 mg/kg, the threshold exposure level can range from 5-40 μg/mL (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 μg/mL); if the dose is 10 mg/kg, the threshold exposure level can range from 20-120 μg/mL (20, 25, 30, 35, 40, 45, 50, 55, 60, 65, 70, 75, 80, 85, 90, 95, 100, 105, 110, 115 or 120 μg/mL).

The terms “about” or “approximately” mean within an acceptable range for the particular parameter specified as determined by one of ordinary skill in the art, which will depend in part on how the value is measured or determined, e.g., the limitations of the measurement system. For example, “about” can mean a range of up to 20% of a given value. Alternatively, particularly with respect to biological systems or processes, the term can mean within an order of magnitude, preferably within 5-fold, and more preferably within 2-fold, of a value.

The terms “cytotoxic T lymphocyte-associated antigen-4,” “CTLA-4,” “CTLA4,” “CTLA-4 antigen” and “CD152” (see, e.g., Murata (1999) Am. J. Pathol. 155:453-460) are used interchangeably, and include variants, isoforms, species homologs of human CTLA-4, and analogs having at least one common epitope with CTLA-4 (see, e.g., Balzano (1992) Int. J. Cancer Suppl. 7:28-32). A complete sequence of human CTLA-4 is set forth in GenBank Accession No. L15006.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

### CTLA4 Antibodies

In certain aspect, the present invention relates to therapeutic use of anti-CTLA4 antibodies. Preferably, the anti-CTLA4 antibody used in the methods is a human antibody. More preferably, the anti-CTLA4 antibody is MDX-010 (also referred to as ipilimumab or Yervoy). An exemplary anti-CTLA4 antibody comprises: (a) a heavy chain variable region comprising the amino acid sequence of SEQ ID NO: 17; and (b) a light chain variable region comprising the amino acid sequence of SEQ ID NO: 7. Another exemplary anti-CTLA4 antibody comprises: (a) a heavy chain variable region CDR1 comprising SEQ ID NO: 27; (b) a heavy chain variable region CDR2 comprising SEQ ID NO: 32; (c) a heavy chain variable region CDR3 comprising SEQ ID NO: 37; (d) a light chain variable region CDR1 comprising SEQ ID NO: 24; (e) a light chain variable region CDR2 comprising SEQ ID NO: 29; and (f) a light chain variable region CDR3 comprising SEQ ID NO: 35.

The term “antibody”, as referred to herein, includes antigen-binding portions of an intact antibody that retain capacity to bind CTLA-4. Examples of binding include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 1998; 242:423-426; and Huston et al., Proc. Natl. Acad. Sci. USA 1988; 85:5879-5883). Such single chain antibodies are included by reference to the term “antibody.” Fragments can be prepared by recombinant techniques or enzymatic or chemical cleavage of intact antibodies.

The term “human sequence antibody” or “human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Such antibodies can be generated in non-human transgenic animals, i.e., as described in PCT Publication Nos. WO 01/14424 and WO 00/37504. However, the term “human sequence antibody” or “human antibody”, as used herein, is not intended to include antibodies in which CDR sequences derived from the germline of another mammalian species, such as a mouse, have been grafted onto human framework sequences (i.e., humanized antibodies).

The terms “monoclonal antibody” or “monoclonal antibody composition” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

CTLA-4 antibodies can bind to an epitope on human CTLA-4 so as to inhibit CTLA-4 from interacting with a human B7 counterreceptor. Because interaction of human CTLA-4 with human B7 transduces a signal leading to inactivation of T-cells bearing the human CTLA-4 receptor, antagonism of the interaction effectively induces, augments or prolongs the activation of T cells bearing the human CTLA-4 receptor, thereby prolonging or augmenting an immune response. CTLA-4 antibodies are described in U.S. Pat. Nos. 5,811,097, 5,855,887, 6,051,227, 6,984,720, 7605238; in PCT Publication Nos. WO 01/14424 and WO 00/37504; and in U.S. Publication No. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., PNAS 1998; 95(17):10067-10071; Camacho et al., J Clin Oncology 2004:22(145): abstract no. 2505 (antibody CP-675206); and Mokyr, et al., Cancer Research 1998; 58:5301-5304. Each of these references is specifically incorporated herein by reference for purposes of description of CTLA-4 antibodies. A preferred clinical CTLA-4 antibody is human monoclonal antibody 10D1 (also referred to as MDX-010, ipilimumab, Yervoy, and available from Medarex, Inc., Bloomsbury, N.J.) as disclosed in WO 01/14424.

Also included in the invention are modified antibodies. The term “modified antibody” includes antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody.

For example, anti-CTLA4 antibodies of the invention include antibodies whose heavy and light chain variable regions comprising amino acid sequences that are homologous to the amino acid sequences of the preferred antibodies described herein, and wherein the antibodies retain the desired functional properties of the anti-CTLA4 antibodies of the invention. For example, an anti-CTLA4 antibody includes an antibody comprising: (a) the heavy chain variable region comprises an amino acid sequence that is at least 80%, 90%, 95%, 98%, or 99% identical to an amino acid sequence selected from the group consisting of SEQ ID NO: 17; (b) the light chain variable region comprises an amino acid sequence that is at least 80%, 90%, 95%, 98%, or 99% identical to an amino acid sequence selected from the group consisting of SEQ ID NOs: 7. In another example, an anti-CTLA4 antibody includes an antibody comprising: (a) a heavy chain variable region CDR1 comprising an amino acid sequence that is at least 80%, 90%, 95%, 98%, or 99% identical to SEQ ID NO: 27; (b) a heavy chain variable region CDR2 comprising an amino acid sequence that is at least 80%, 90%, 95%, 98%, or 99% identical to SEQ ID NO: 32; (c) a heavy chain variable region CDR3 comprising an amino acid sequence that is at least 80%, 90%, 95%, 98%, or 99% identical to SEQ ID NO: 37; (d) a light chain variable region CDR1 comprising an amino acid sequence that is at least 80%, 90%, 95%, 98%, or 99% identical to SEQ ID NO: 24; (e) a light chain variable region CDR2 comprising an amino acid sequence that is at least 80%, 90%, 95%, 98%, or 99% identical to SEQ ID NO: 29; and (f) a light chain variable region CDR3 comprising an amino acid sequence that is at least 80%, 90%, 95%, 98%, or 99% identical to SEQ ID NO: 35.

Antibody conjugates are also contemplated for use in the methods of this invention and can be used to modify a given biological response or create a biological response (e.g., to recruit effector cells). An “antibody conjugate,” or “immunoconjugate,” as used herein, is a CTLA-4 antibody conjugated to a drug moiety, such as a cytotoxin, a drug (e.g., an immunosuppressant) or a radiotoxin. The drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-alpha; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Immunoconjugates that include one or more cytotoxins are referred to as “immunotoxins.” A cytotoxin or cytotoxic agent includes any agent that is detrimental to (e.g., kills) cells. Examples include TAXOL®, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (MYLOTARG®; Wyeth-Ayerst).

Also included in the invention are bispecific molecules comprising an anti-CTLA4 antibody or a fragment thereof. An anti-CTLA4 antibody or antigen-binding portions thereof, can be derivatized or linked to another functional molecule, e.g., another peptide or protein (e.g., another antibody or ligand for a receptor) to generate a bispecific molecule that binds to at least two different binding sites or target molecules. An anti-CTLA4 antibody or a fragment thereof may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule, an anti-CTLA4 antibody can be functionally linked (e.g., by chemical coupling, genetic fusion, noncovalent association or otherwise) to one or more other binding molecules, such as another antibody, antibody fragment, peptide or binding mimetic, such that a bispecific molecule results.

### Methods of Detection Assays

In certain embodiments, the present invention provides a method for detecting a therapeutic anti-CTLA4 antibody (e.g., MDX-010) in a sample from a subject. For example, a body fluid (e.g., blood, serum or plasma) or tissue sample from the test subject is contacted with an anti-MDX-010 monoclonal antibody, or antigen binding portion thereof, of the invention under conditions suitable for the formation of antibody-antigen complexes. The presence or amount of such complexes can then be determined by methods described herein and otherwise known in the art (see, e.g., O'Connor et al., Cancer Res., 48:1361-1366 (1988)), in which the presence or amount of complexes found in the test sample is compared to the presence or amount of complexes found in a series of standards or control samples containing a known amount of antigen. Accordingly, the present invention relates to methods for detecting an anti-CTLA4 antibody (such as MDX-010) in a biological sample (e.g., blood, serum, plasma, urine, cerebrospinal fluid, mucus, or saliva).

In any of the described detection assays, the method can employ an immunoassay, e.g., an enzyme immunoassay (EIA), enzyme-linked immunosorbant assay (ELISA), radioimmunoassay (RIA), indirect competitive immunoassay, direct competitive immunoassay, non-competitive immunoassay, sandwich immunoassay, agglutination assay or other immunoassay describe herein and known in the art (see, e.g., Zola, Monoclonal Antibodies: A Manual of Techniques, pp. 147-158, CRC Press, Inc. (1987)). Immunoassays may be constructed in heterogeneous or homogeneous formats. Heterogeneous immunoassays are distinguished by incorporating a solid phase separation of bound analyte from free analyte or bound label from free label. Solid phases can take a variety of forms well known in the art, including but not limited to tubes, plates, beads, and strips. One particular form is the microtiter plate. The solid phase material may be comprised of a variety of glasses, polymers, plastics, papers, or membranes. Particularly desirable are plastics such as polystyrene. Heterogeneous immunoassays may be competitive or non-competitive (i.e., sandwich formats) (see, e.g., U.S. Pat. No. 7,195,882).

In a specific embodiment, the detection method of the present invention includes the following steps (see below).

In the first step of the assay, a biological sample isolated from a subject is contacted and incubated with an immobilized capture antigen (such as a CTLA4 protein). Immobilization may be accomplished by insolubilizing the capture antigen either before the assay procedure, as by adsorption to a water-insoluble matrix or surface (U.S. Pat. No. 3,720,760) or non-covalent or covalent coupling (for example, using glutaraldehyde or carbodiimide cross-linking, with or without prior activation of the support with, e.g., nitric acid and a reducing agent as described in U.S. Pat. No. 3,645,852 or in Rotmans et al., J. Immunol. Methods, 57:87-98 (1983)), or afterward, e.g., by immunoprecipitation.

The solid phase used for immobilization may be any inert support or carrier that is essentially water insoluble and useful in immunometric assays, including supports in the form of, e.g., surfaces, particles, porous matrices, etc. Examples of commonly used supports include small sheets, SEPHADEX® gels, polyvinyl chloride, plastic beads, and assay plates or test tubes manufactured from polyethylene, polypropylene, polystyrene, and the like, including 96-well microtiter plates, as well as particulate materials such as filter paper, agarose, cross-linked dextran, and other polysaccharides. Alternatively, reactive water-insoluble matrices such as cyanogens-bromide-activated carbohydrates and the reactive substrates described in U.S. Pat. Nos. 3,969,287; 3,691,016; 4,195,128; 4,247,642; 4,229,537; and 4,330,440 are suitably employed for capture-reagent immobilization. In a specific embodiment, the immobilized capture antigen is coated on a microtiter plate, and in particular the solid phase used is a multi-well microtiter plate that can be used to analyze several samples at one time. The most preferred is a MICROTEST® or MaxiSorp 96-well ELISA plate such as that sold as NUNC® MaxiSorb or IMMULON®. The solid phase is coated with the capture antigen, which may be linked by a non-covalent or covalent interaction or physical linkage as desired. Techniques for attachment include those described in U.S. Pat. No. 4,376,110 and the references cited therein. If covalent, the plate or other solid phase is incubated with a cross-linking agent together with the capture antibody under conditions well known in the art such as for one hour at room temperature. Commonly used cross-linking agents for attaching the capture reagents to the solid-phase substrate include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-((p-azidophenyl)-dithio)propioimidate yield photoactivatable intermediates capable of forming cross-links in the presence of light.

The coated plates may then be treated with a blocking agent that binds non-specifically to and saturates the binding sites to prevent unwanted binding of the free ligand to the excess sites on the wells of the plate. Examples of appropriate blocking agents for this purpose include, e.g., gelatin, bovine serum albumin, egg albumin, casein, and non-fat milk. The blocking treatment can take place under conditions of ambient temperatures for about 1-4 hours, preferably about 1.5 to 3 hours.

The conditions for incubation of sample and immobilized capture antigen are selected to maximize sensitivity of the assay and to minimize dissociation, and to ensure that any anti-CTLA4 antibody in the sample binds to the immobilized capture antigen. Preferably, the incubation is accomplished at fairly constant temperatures, ranging from about 0° C. to about 40° C., preferably at or about room temperature. The time for incubation is generally no greater than about 10 hours. Preferably, the incubation time is from about 0.5 to 3 hours, and more preferably about 1.5-3 hours at or about room temperature to maximize binding of the antibody of interest to the capture antibody. The duration of incubation may be longer if a protease inhibitor is added to prevent proteases in the biological fluid from degrading the anti-CTLA4 antibody.

In a second step of the assay method herein, which is optional, the biological sample is separated (preferably by washing) from the immobilized capture antigen to remove the uncaptured anti-CTLA4 antibody (e.g., MDX-010). The washing may be done three or more times. The temperature of washing is generally from refrigerator to moderate temperatures, with a constant temperature maintained during the assay period, typically from about 0-40° C., more preferably about 4-30° C. A cross-linking agent or other suitable agent may also be added at this stage to allow the now-bound antibody to be covalently attached to the capture antigen if there is any concern that the captured anti-CTLA4 antibody may dissociate to some extent in the subsequent steps.

In the third step, the immobilized capture antigen with any bound anti-CTLA 4 antibody (e.g., MDX-010) are contacted with a detectable antibody, preferably at a temperature of about 20-40° C., more preferably about 36-38° C. The detectable antibody may be a polyclonal or monoclonal antibody. Optionally it is a monoclonal antibody, such as a rodent monoclonal antibody or a murine monoclonal antibody. Optionally, the detectable antibody is directly detectable, and such as biotinylated. The detection means for the biotinylated label is preferably avidin or streptavidin-HRP, and the readout of the detection means is preferably fluorimetric or colorimetric.

In the fourth step of the assay method, the level of any free antibody of interest (e.g., MDX-010) from the sample that is now bound to the capture antigen is measured using a detection means for the detectable antibody. If the biological sample is from a clinical patient, the measuring step preferably comprises comparing the reaction that occurs as a result of the above three steps with a standard curve to determine the level of antibody of interest compared to the known amount.

The detectable antibody (herein referred to as the “first antibody”) will be either directly labeled, or detected indirectly by addition, after washing off of excess first antibody, of a molar excess of a second, labeled antibody directed against IgG of the animal species of the first antibody. In the latter, indirect assay, labeled antisera against the first antibody are added to the sample so as to produce the labeled antibody in situ. The label used for either the first or second antibody is any detectable functionality that does not interfere with the binding of free antibody of interest to the capture antigen. Examples of suitable labels are those numerous labels known for use in immunoassay, including moieties that may be detected directly, such as fluorochrome, chemiluminscent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare-earth chelates or fluorescein and its derivatives, rhodamine and its derivatives, dansyl, umbelliferone, luceriferases, e.g., firefly luciferase and bacterial luciferase (see, e.g., U.S. Pat. No. 4,737,456), luciferin, 2,3-dihydrophthalazinediones, HRP, alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin (detectable by, e.g., avidin, streptavidin, streptavidin-HRP, and streptavidin-β-galactosidase with MUG), spin labels, bacteriophage labels, stable free radicals, and the like. In a specific embodiment, the label is biotin and the detection means is avidin or streptavidin-HRP.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the antibodies with the above-described fluorescent, chemiluminescent, and enzyme labels. See, e.g., U.S. Pat. No. 3,940,475 (fluorimetry) and U.S. Pat. No. 3,645,090 (enzymes); Hunter et al., Nature, 144:945 (1962); David et al., Biochemistry, 13:1014-1021 (1974); Pain et al., J. Immunol. Methods, 40:219-230 (1981); and Nygren, J. Histochem. Cytochem., 30:407-412 (1982). An exemplary label is biotin using streptavidin-HRP for detection means. The conjugation of such label, including the enzymes, to an antibody is a standard manipulative procedure for one of ordinary skill in immunoassay techniques. See, e.g., O'Sullivan et al. “Methods for the Preparation of Enzyme-antibody Conjugates for Use in Enzyme Immunoassay,” in Methods in Enzymology, Langone, J. J. and Van Vunakis, H., eds. Vol. 73, pp. 147-166, Academic Press, New York, N.Y. (1981).

Following the addition of last labeled antibody, the amount of bound antibody is determined by removing excess unbound labeled antibody through washing and then measuring the amount of the attached label using a detection method appropriate to the label, and correlating the measured amount with the amount of the antibody of interest in the biological sample. For example, in the case of enzymes, the amount of color developed and measured will be a direct measurement of the amount of the antibody of interest present. Specifically, if HRP is the label, the color is detected using the substrate OPD at 490-nm absorbance. In another example, after an enzyme-labeled second antibody directed against the first unlabeled antibody is washed from the immobilized phase, color or chemiluminiscence is developed and measured by incubating the immobilized capture reagent with a substrate of the enzyme. Then the concentration of the antibody of interest is calculated by comparing with the color or chemiluminescence generated by the standard antibody of interest run in parallel.

### Kits

In certain embodiments, the present invention provides kits that can be used in the detection assays described above, which comprise an antigen which binds an anti-CTLA4 antibody (e.g., MDX-010) as well as reagents necessary for facilitating an antibody-antigen complex formation and/or detection. Preferably, an antigen of such kits is a CTLA4 protein (e.g., a CTLA4/Fc fusion protein). For example, a kit of the present invention is a packaged combination including the basic elements of: (a) capture reagents comprising at least one antigen which binds an anti-CTLA4 antibody (herein referred to as a “capture antigen”); and (b) instructions on how to perform the assay method using these reagents.

Optionally, the kit further comprises a solid support for the capture antigen, which may be provided as a separate element or on which the capture antigen is already immobilized. Hence, the capture antigen in the kit may be immobilized on a solid support, or may be immobilized on such support that is included with the kit or provided separately from the kit. For example, the capture antigen is coated on a microtiter plate. Optionally, the kit further comprises at least one detectable antibody. The detectable antibodies may be labeled antibodies detected directly or unlabeled antibodies that are detected by labeled antibodies directed against the unlabeled antibodies raised in a different species. Where the label is an enzyme, the kit will ordinarily include substrates and cofactors required by the enzyme, where the label is a fluorophore, a dye precursor that provides the detectable chromophore, and where the label is biotin, an avidin such as avidin, streptavidin, or streptavidin conjugated to HRP or β-galactosidase with MUG.

The kit may further comprise, as a positive control, the antibody of interest (e.g., purified MDX-010). The kits may further comprise, as a negative control, an antibody which does not react with the capture antigen. The kit may further comprise other additives such as stabilizers, washing and incubation buffers, and the like. The components of the kit will be provided in predetermined ratios, with the relative amounts of the various reagents suitably varied to provide for concentrations in solution of the reagents that substantially maximize the sensitivity of the assay. Particularly, the reagents may be provided as dry powders, usually lyophilized, including excipients, which on dissolution will provide for a reagent solution having the appropriate concentration for combining with the sample to be tested.

### Therapeutic Methods

The present invention relates to methods of treating CTLA4-related diseases which include, for example, cancers, infectious diseases, and diseases caused by an inappropriate accumulation of self-antigens.

Cancers

The terms “cancer” and “tumor” are used herein interchangeably. The present invention is directed, in part, to the treatment of tumors, particularly immunologically sensitive tumors, which are cancers that respond to immunotherapy or cancers that manifest in patients who are immunocompromised. A tumor treated with the methods of this invention can be a solid tumor.

Examples of tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, lymphoma, melanoma, Kaposi's sarcoma, Ewing's tumor, leiomyosarcoma, rhabdomyosarcoma, colo-rectal carcinoma, gastric carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The methods of this invention can also treat or prevent dysproliferative changes (such as metaplasias and dysplasias) in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

The present invention is also directed to treatment of non-malignant tumors and other disorders involving inappropriate cell or tissue growth augmented by angiogenesis by administering a therapeutically effective amount of a vector of the invention to the tissue undergoing inappropriate growth. For example, it is contemplated that the invention is useful for the treatment of arteriovenous (AV) malformations, particularly in intracranial sites. The invention may also be used to treat psoriasis, a dermatologic condition that is characterized by inflammation and vascular proliferation; and benign prostatic hypertrophy, a condition associated with inflammation and possibly vascular proliferation. Treatment of other hyperproliferative disorders is also contemplated.

The term “advanced cancer” means cancer that is no longer localized to the primary tumor site, or a cancer that is Stage III or IV according to the American Joint Committee on Cancer (AJCC).

Specific examples of the cancer include, but are not limited to, melanoma, prostate cancer, lung cancer, gastric cancer, ovarian cancer, breast cancer, and glioblastoma.

Infectious Diseases

Other methods of the invention are used to treat patients that have been exposed to pathogens. Similar to its application to tumors as discussed above, CTLA-4 antibodies administered according to a dosage escalation regimen of the present invention can be used alone, or in combination with a vaccine to treat an infectious disease. CTLA-4 blockade has been shown to be effective in the acute phase of infections of Nippostrongylus brasiliensis (McCoy, K. et al. (1997) 186(2); 183-187) and Leishmania donovani (Murphy, M. et al. (1998) J. Immunol. 161:4153-4160). Examples of pathogens for which this therapeutic approach may be particularly useful include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, and Pseudomonas aeruginosa. CTLA-4 blockade is particularly useful in boosting immunity against established infections by agents such as HIV that present altered antigens over the course of the infections. These novel epitopes are recognized as foreign at the time of anti-human CTLA-4 administration, thus provoking a strong T-cell response that is not dampened by negative signals through CTLA-4.

Some examples of pathogenic viruses causing infections treatable by methods of the invention include hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods of the invention include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (Mucor, Absidia, Rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, Nippostrongylus brasiliensis.

Diseases Cause by Inappropriate Accumulation of Self-Antigens

A CTLA-4 antibody can be administered according to the present invention to treat a patient having an inappropriate accumulation of self-antigens, such as amyloid deposits, cytokines such as TNF-alpha, and IgE (for the treatment of allergy and asthma). For example, Alzheimer's disease involves inappropriate accumulation of Aβ peptide in amyloid deposits in the brain; antibody responses against amyloid are able to clear these amyloid deposits (Schenk et al., Nature 1999; 400:173-177).

### Combination Treatments

CTLA-4 Antibodies and Vaccines for the Treatment of Cancer

According to the methods of the present invention, a CTLA-4 antibody can be administered alone or in combination with one or more other therapeutic agents, or in conjunction with an immunotherapeutic vaccine for the tumor, such as chemotherapy, radiation therapy, cytokines, chemokines and other biologic signaling molecules, tumor specific vaccines, autologous and allogeneic stem cell rescue (e.g., to augment graft versus tumor effects), other therapeutic antibodies, molecular targeted therapies, anti-angiogenic therapy, infectious agents with therapeutic intent (such as tumor localizing bacteria), and gene therapy. The antibodies can be used in adjuvant or neoadjuvant therapy, either alone or in conjunction with the aforementioned therapies.

Antibodies to CTLA-4 can be combined with an immunogenic agent, such as cancerous cells, purified tumor antigens (including recombinant proteins, peptides, and carbohydrate molecules), cells, and cells transfected with genes encoding immune stimulating cytokines and cell surface antigens such as B7 (see, e.g., Hurwitz, A. et al. (1998) Proc. Natl. Acad. Sci. U.S.A. 1998; 95:10067-10071), or used alone, to stimulate immunity.

Treatment with a CTLA-4 antibody can be used to activate a pre-existing memory response in patients treated with a cancer vaccine. Thus, methods of this invention include treating vaccine-treated patients who are selected for further treatment with a CTLA-4 antibody to thereby further induce or enhance an immune response.

Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. Proc. Natl. Acad. Sci. U.S.A. 1993; 90: 3539-43).

CTLA-4 blockade to boost GMCSF-modified tumor cell vaccines improves efficacy of vaccines in a number of experimental tumor models such as mammary carcinoma (Hurwitz et al., 1998, supra), primary prostate cancer (Hurwitz et al., Cancer Research 2000; 60:2444-8) and melanoma (van Elsas et al. J. Exp. Med. 1999, 190:355-66). In these instances, non-immunogenic tumors, such as the B16 melanoma, have been rendered susceptible to destruction by the immune system. The tumor cell vaccine may also be modified to express other immune activators such as IL-2, and costimulatory molecules, among others.

The study of gene expression and large scale gene expression patterns in various tumors has led to the definition of so called “tumor specific antigens” (Rosenberg, Immunity 1999; 10:281-7). In many cases, these tumor specific antigens are differentiation antigens expressed in the tumors and in the cell from which the tumor arose, for example melanocyte antigens gp100, MAGE, and Trp-2. More importantly, many of these antigens can be shown to be the targets of tumor specific T-cells found in the host. CTLA-4 blockade may be used as a boosting agent in conjunction with vaccines based on recombinant versions of proteins and/or peptides found to be expressed in a tumor. The tumor antigen may also include the protein telomerase, which is required for the synthesis of telomeres of chromosomes and which is expressed in more than 85% of human cancers and in only a limited number of somatic tissues (Kim et al., Science 1994; 266:2011-2013). These somatic tissues may be protected from immune attack by various means.

Tumor antigen may also be “neo-antigens” expressed in cancer cells because of somatic mutations that alter protein sequence or create fusion proteins between two unrelated sequences (i.e. bcr-abl in the Philadelphia chromosome), or idiotype from B-cell tumors. Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with CTLA-4 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot and Srivastava, Science 1995; 269:1585-1588; Tamura et al., Science 1997, 278:117-120).

Dendritic cells (DC) are potent antigen presenting cells that can be used to prime antigen-specific responses. DC's can be produced ex vivo and loaded with various protein and peptide antigens as well as tumor cell extracts (Nestle et al., Nature Medicine 1998; 4:328-332). DCs may also be transduced by genetic means to express these tumor antigens as well. DCs have also been fused directly to tumor cells for the purposes of immunization (Kugler et al., Nature Medicine 2000; 6:332-336). As a method of vaccination, DC immunization may be effectively boosted with CTLA-4 blockade according to a dosage escalation regimen of the present invention to activate more potent anti-tumor responses.

Another type of melanoma vaccine that can be combined with CTLA-4 blockade according to the present invention is a vaccine prepared from a melanoma cell line lysate, in conjunction with an immunological adjuvant, such as the MELACINE® vaccine, a mixture of lysates from two human melanoma cell lines plus DETOX™ immunological adjuvant. Vaccine treatment can be boosted with CTLA-4 antibody, with or without additional chemotherapeutic treatment.

Chemotherapeutic Agents and Other Standard Cancer Treatments

CLTA-4 administration according to the present invention can be used in combination with standard cancer treatments. In these instances, it may be possible to reduce the dose of chemotherapeutic reagent administered (Mokyr et al., Cancer Research, 1998; 58:5301-5304). The scientific rationale behind the combined use of CTLA-4 blockade and chemotherapy is that cell death, that is a consequence of the cytotoxic action of most chemotherapeutic compounds, should result in increased levels of tumor antigen in the antigen presentation pathway. Thus, CTLA-4 can boost an immune response primed to chemotherapy release of tumor cells. Moreover, the immuno-stimulatory activity of CTLA-4 is useful to overcome the immunosuppressive effects of chemotherapy. Examples of chemotherapeutic agents with which CTLA-4 treatment can be combined include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, topotecan hydrochloride, trastuzumab, vinblastine, vincristine and vinorelbine tartrate. For prostate cancer treatment, a preferred chemotherapeutic agent with which CTLA-4 can be combined is paclitaxel (Taxol®). For melanoma cancer treatment, a preferred chemotherapeutic agent with which CTLA-4 can be combined is dacarbazine (DTIC).

Other combination therapies that may result in immune system priming through cell death are radiation, surgery, and hormone deprivation (Kwon, E. et al. Proc. Natl. Acad. Sci. U.S.A. 1999; 96 (26): 15074-9. Each of these protocols creates a source of tumor antigen in the host. For example, any manipulation of the tumor at the time of surgery can greatly increase the number of cancer cells in the blood (Schwartz, et al., Principles of Surgery 1984. 4th ed. p. 338). Angiogenesis inhibitors may also be combined with a CTLA-4 antibody dosage escalation regimen. Inhibition of angiogenesis leads to tumor cell death which may feed tumor antigen into host antigen presentation pathways.

Cytokines

A CTLA-4 antibody administered in a dosage escalation regimen according to the present invention can also be combined with other forms of immunotherapy such as cytokine treatment (e.g., interferons, GMCSF, GCSF, IL-2, or bispecific antibody therapy, which provides for enhanced presentation of tumor antigens (see e.g., Holliger (1993) Proc. Natl. Acad. Sci. USA 90:6444-6448; Poljak (1994) Structure 2:1121-1123). For example, dosages regimens for cytokines include 720,000 IU/kg/dose every 8 hours for up to 15 doses per dosage of CTLA-4 antibody.

### Pharmaceutical Compositions and Routes of Administration

In certain embodiments, the invention encompasses pharmaceutical compositions comprising a CTLA-4 human monoclonal antibody (intact or binding fragments) formulated together with a pharmaceutically acceptable carrier for use in a dosage escalation regimen. Some compositions include a combination of multiple (e.g., two or more) isolated human CTLA-4 antibodies and/or human sequence antibody or antigen-binding portions thereof of the invention.

Pharmaceutically acceptable carriers include solvents, dispersion media, coatings, antibacterial and antifungal agents (e.g., paraben, chlorobutanol, phenol sorbic acid, and the like), isotonic and absorption delaying agents, and the like that are physiologically compatible. The carrier can be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

Pharmaceutically acceptable carriers also include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27). Supplementary active compounds can also be incorporated into the compositions.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof, vegetable oils, such as olive oil, and injectable organic esters, such as ethyl oleate. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. These compositions may also contain adjuvants such as preservatives, wetting agents, emulsifying agents and dispersing agents.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

A “pharmaceutically acceptable salt” refers to a salt that retains the desired biological activity of the parent compound and does not impart any undesired toxicological effects (See e g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition for use in a dosage escalation regimen according to the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, 1978. In addition, prolonged absorption of an injectable pharmaceutical form may be brought about by the inclusion of agents which delay absorption such as aluminum monostearate and gelatin. Pharmaceutical compositions are preferably manufactured under GMP conditions.

Examples of pharmaceutically-acceptable antioxidants for use in pharmaceutical compositions include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations for use in the methods of the present invention include those suitable for oral, nasal, topical (including buccal and sublingual), rectal, vaginal and/or parenteral administration. The formulations can conveniently be presented in unit dosage form and may be prepared by any methods known in the art of pharmacy. The amount of active ingredient which can be combined with a carrier material to produce a single dosage form varies depending upon the subject being treated, and the particular mode of administration. Generally, out of one hundred percent, this amount ranges from about 0.01 percent to about ninety-nine percent of active ingredient, from about 0.1 percent to about 70 percent, or from about 1 percent to about 30 percent.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions can be administered with medical devices known in the art. For example, in a preferred embodiment, a therapeutic composition of the invention can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

Some human sequence antibodies and human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (See, e.g., V. V. Ranade (1989) J. Clin. Pharmacol. 29:685). Exemplary targeting moieties include folate or biotin (See, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); See also K. Keinanen; M. L. Laukkanen (1994) FEBS Lett. 346:123; J. J. Killion; I. J. Fidler (1994) Immunomethods 4:273. In some methods, the therapeutic compounds of the invention are formulated in liposomes; in a more preferred embodiment, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

The present invention is further illustrated by the following examples which should not be construed as further limiting. The contents of all figures and all references, patents and published patent applications cited throughout this application are expressly incorporated herein by reference.

### EXAMPLE 1

### Ipilimumab Population Pharmacokinetic and Exposure-Response Analyses in Previously Treated or Untreated Subjects with Advanced Melanoma

### Data

The population pharmacokinetic (PPK) analyses was performed with PK data from 29 subjects in one chemo-combination phase 1 study (CA184078), four phase 2 monotherapy clinical studies (CA184004 [79 subjects], CA184007 [112 subjects], CA184008 [148 subjects] and CA184022 [177 subjects]) and 240 subjects in one randomized phase 3 study in combination with dacarbazine (CA184024). All subjects had advanced melanoma. Altogether there were 785 subjects who contributed 3200 samples to the PPK dataset. Of these, 528 study subjects received ipilimumab alone, while 257 received ipilimumab with dacarbazine. The exposure-response (E-R) analysis for OS was performed with data from study CA184024, a phase 3 study in subjects with previously untreated advanced melanoma. The E-R analyses for irAEs were performed with data from all the studies included in the population pharmacokinetic (PPK) analysis.

These studies were selected for inclusion in the PPK and E-R analysis on the basis of the available PK data, covariates, and response endpoints of interest. The four phase 2 studies were included in an earlier PPK and E-R analyses that focused on previously treated advanced melanoma patients. The inclusion of data from studies CA184024 and CA184078 in the current PPK analysis was to enable a robust characterization of ipilimumab PK in previously untreated advanced melanoma patients, and the evaluation of the effect of concomitant dacarbazine on the PK of ipilimumab. Data from studies CA184024 and CA184078 were also used to characterize E-R in previously untreated advanced melanoma patient population.

All 498 subjects in Study CA180024 were included in the OS E-R analysis dataset. The ipilimumab exposure of subjects in the placebo (dacarbazine with placebo) group was assumed to be zero.

The analysis dataset for characterization of the ipilimumab exposure-irAE relationship comprised 1036 subjects; 785 administered doses of 0.3, 3, or 10 mg/kg ipilimumab, and 251 subjects administered placebo. The response variables were categorized worst-grade irAEs of Grade>=2 and Grade>=3 for gastrointestinal, hepatobiliary, skin or any-type irAE. The exposure metric was the subject-specific estimates of steady-state trough concentration (Cminss) obtained from the population PK model and the empirical Bayesian estimates of subject-specific PK parameters.

### Methods

### 1. Population Pharmacokinetic Analysis

The PPK model was developed in three stages. The first step was to confirm the appropriateness of the base PPK model that was previously developed to describe the PK of ipilimumab. This was done without considering covariate effects. In the second stage, a full-covariate model was developed incorporating the effect of all pre-specified covariate-parameter relationships. In the third stage, the final PPK model was developed by retaining not only covariates that improved the goodness-of-fit statistic (Bayesian Information Criterion [BIC]) and were of potential clinical relevance.

Baseline covariates examined were body weight, age, gender, estimated glomerular filtration (eGFR) rate, Eastern Oncology Group (ECOG) performance status, baseline lactate dehydrogenase (LDH), dacarbazine, prior systemic anti-cancer therapy. In addition, the effect of immunogenicity on clearance was assessed as a time-varying covariate to account for the possibility that human anti-human antibodies (HAHA) are not present at all times in immunogenic subjects. Covariate models were developed for ipilimumab clearance and central volume of distribution. No covariates were modeled on the peripheral volume of distribution and intercompartmental clearance.

Visual predictive check with and without bias correction was used to evaluate the prediction performance of the developed final PPK model, given the data. The final PPK model was used to predict steady-state ipilimumab steady trough concentration (Cminss) for exposure-response (E-R) analyses.

### 2. Exposure-Efficacy Response Analysis: OS

The exposure-overall survival relationship was characterized with a Cox proportional-hazards (CPH) model relating Cminss to the hazard of death. The CPH model was developed in 3 stages. First, a base model was developed to establish the existence and functional form of the E-R relationship between OS and ipilimumab Cminss. Second, a full model was developed to assess the effect of all of the potential covariates of interest. Third, the final model was developed by retaining potentially clinically relevant predictors, with appropriate functional forms of their relationships with OS, using the BIC. The CPH model was evaluated by comparing model predicted cumulative probability of OS versus time with that obtained with Kaplan-Meier (KM) analysis.

3. Exposure-Safety Response Analyses: irAE

The relationship between Cminss and the probability of irAEs was modeled using a proportional odds logistic regression with an Emax model describing the relationship between Cminss and the logit-transformed probability. A separate model was developed for each irAE type; gastroinestinal, hepatobiliary, skin, and “any”. Prespecified candidate covariates were collected into a full model that was subject to a stepwise backward elimination procedure governed by the BIC to realize a parsimonious model. The performance of the models was evaluated through diagnostic plots as well as prediction check methods.

### Results

### I. Population Pharmacokinetic Analysis

### The PPK Model Provided an Adequate Description of Ipilimumab Concentration-Time Data in the Target Population.

The PPK model for ipilimumab was developed and evaluated using 3200 observations of serum concentration from 785 subjects with advanced melanoma. Ipilimumab PK was described with a linear two-compartment model with zero-order IV infusion parameterized in terms of clearance (CL), volume of central compartment (VC), inter-compartmental clearance (Q), and volume of peripheral compartment (VP). Interindividual variability in CL and VC were characterized with lognormal distributions, and a combined additive and proportional error model was used to characterize the residual error.

Covariate analysis revealed that baseline body weight and LDH were potentially clinically relevant predictors of CL, and body weight a significant predictor of VC. Ipilimumab CL was determined to increase with body weight (BW) and LDH. Subjects with poor ECOG performance status (ECOG=1) had slightly higher CL (i.e., 16% higher than those with ECOG=0), but the magnitude of the effect was unlikely to be of clinical relevance. Central volume of distribution for ipilimumab increases with increase in body weight. Gender and eGFR were retained in the final model for CL because of the improvement in the goodness-of-fit-statistic, but they are of no clinical relevance. The same is true for gender on VC. Concomitant dacarbazine, prior systemic anti-cancer therapy, and immunogenicity status were not retained in the final model because they were found not to relevant clinical predictors of ipilimumab PK. The final PPK model, given the data, describing the covariate effects on the typical values (model estimated geometric mean) of CL and VC are described as follows:

The final model was as follows:

$${CL}_{TV} = {{CL}_{REF} ( \frac{BW}{{BW}_{REF}} )}^{{CL}_{BW}} {( \frac{log ( LDH )}{log ( {LDH}_{REF} )} )}^{{CL}_{LDH}} {( \frac{GFR}{{GFR}_{REF}} )}^{{CL}_{GFR}} {( {CL}_{Sex} )}^{{CL}_{SEX}} {( {CL}_{ECOG} )}^{{CL}_{ECOG}}$$

$${VC}_{TV} = {{VC}_{REF} ( \frac{BW}{{BW}_{REF}} )}^{{VC}_{BW}} {( {VC}_{Sex} )}^{{VC}_{SEX}}$$

where CL_{REF} and VC_{REF} are the typical values (model-estimated geometric mean) of CL and VC at the reference values of BW, LDH, and GFR, respectively, and gender is referenced to male and ECOG performance status to 0. CL_{BW}, CL_{LDH}, CL_{GFR}, CL_{Sex}, CL_{ECOG}, VC_{BW}, and VC_{Sex} are model parameters. The reference values of BW and LDH were selected to be the median values of these covariates in the PPK dataset.

Following IV infusion, ipilimumab undergoes biphasic elimination. The terminal half-life calculated from the typical values of PK parameters (see Table 1 below) was 15.4 days. The results of the visual predictive check revealed that the model was appropriate for its intended purpose, the generation of steady state trough concentrations for exposure-response analyses.

TABLE 1
Population Parameters for Ipilimumab

| | | |
|---|---|---|
| Parameter [Units] | Parameter Estimate^{a} | 95% CI^{b} |

Fixed Effects

| | | |
|---|---|---|
| CL_{REF} [L/h]^{c} | 0.0153 | (0.0146, 0.161) |
| VC_{REF} [L] | 4.35 | (4.26, 4.44) |
| Q_{REF} [L/h] | 0.0451 | (0.0398, 0.0524) |
| VP_{REF} [L] | 3.28 | (3.10, 3.53) |
| CL_{BW} | 0.580 | (0.427, 0.733) |
| CL_{LDH} | 0.950 | (0.694, 1.214) |
| CL_{eGFR} | 0.290 | (0.204, 0.388) |
| CL_{Sex}, REF = Male | 0.885 | (0.823, 0.951) |
| CL_{ECOG} | 1.16 | (1.096, 1.236) |
| VC_{WT} | 0.534 | (0.448, 0.622) |
| VC_{Sex} | 0.887 | (0.848, 0.922) |

Inter-Individual Variability (IIV)

| | | |
|---|---|---|
| ω²_{CL} | 0.120 (34.6%) | (0.097, 0.141) |
| ω²_{VC} | 0.0221 (14.9%) | (0.0148, 0.0299) |
| ω_{CL}:ωVC | 0.0255 (0.495) | (0.0187, 0.0339) |

Residual Variability^{d}

| | | |
|---|---|---|
| Proportional error | 0.174 | (0.160, 0.188) |
| Additive error | 0.157 | (0.0000295, 0.978) |
| [mcg/mL] | | |

^{a}Estimate values in parentheses are the coefficient of variation for estimated variances and correlation for estimated covariance.
^{b}Confidence Interval values are taken from bootstrap calculations (204 successful out of a total of 700 bootstrap runs).
^{c}Covariate effect was estimated relative to a male reference (typical) subject weighing 79 kg (median value in the PPK dataset), LDH of 204 [IU/L](median), GFR of 86 [mL/min/1.73 m²] (median), and ECOG status of 0.
^{d}Residual variability estimated as standard deviations of the proportional and additive components of the combination error model.

### The Most Influential Covariates on the PK of Ipilimumab Appeared to be Body Weight and LDH.

Body weight and LDH were found to be of potential clinical significance in explaining some variability in ipilimumab CL and VC. The importance of weight on CL is consistent with the mechanism of elimination of full-human monoclonal antibody. Poor ECOG performance status (ECOG=1) was also of some marginal influence on CL. A male subject with a poor ECOG performance status (ECOG=1) has a 16% increase in CL relative to a reference male subject in the PPK dataset. Gender was found to improve the BIC, but it is not a potential clinically relevant predictor of CL. After incorporating these covariates in the final model, the inter-individual variability on CL and VC were reduced by 15.6% and 25.9%, respectively. The estimated variability in the PK parameters did not change in going from the full model to the final model. The PPK analysis showed that both CL and VC increase with body weight. Some data suggests that the body weight normalized dose of ipilimumab is more appropriate for dosing ipilimumab when compared with fixed dosing. It is found that there is a marked downward trend in the Cminss-body weight relationship for a fixed dosage regimen of 800 mg Q3W. The slight increase in exposure for subjects with increase in body weight is due to a less than proportional increase of CL with body weight. Although gender was retained in the final model as a predictor of VC by BIC, it is of no clinical relevance. Its retention may have stemmed from the slight difference in slope between males and females. However, the gender weight difference is taken care of in the weight-based dosing of ipilimumab.

Baseline LDH values less than 900 IU/L (4×ULN) are unlikely to have clinically meaningful impact on ipilimumab clearance. The CL of ipilimumab increased with increasing LDH levels. Ipilimumab CL increased by 26.35% in a male subject with LDH value of 900 IU/L (4×ULN) compared to the reference subject in the PPK dataset. Ipilimumab CL was increased by 30.28% in a male subject with LDH value of 1125 IU/L (5×ULN) compared to the reference subject in the PPK dataset. These results indicate that baseline LDH values less than 900 IU/L are unlikely to have clinically meaningful impact on ipilimumab CL. The increase in CL with increasing baseline LDH has some implications for the efficacy of ipilimumab in melanoma.

LDH is considered a key prognostic factor of reduced survival in advanced melanoma even after accounting for site and number of metastases, and it is used as one of the staging classification factors. Elevated serum LDH may reflect high tumor cell turnover and tumor burden. This might lead to a higher elimination of ipilimumab since ipilimumab CL increases with increasing LDH levels.

Renal Function does not Appear to have any Clinically Relevant Effect on Ipilimumab CL.

Although eGFR was a predictor of CL, its effect on CL was not considered to be of potential clinical relevance. The covariate effect plot for CL reveals that the magnitude of eGFR effect was completely within the ±20% boundaries for the covariate, indicating a lack of clinical relevance. The range of GFR in the dataset covered the normal to severe renal function. Based on the categorization of eGFR using the (Modification in Diet in Renal Disease formula, [MDRD]), 350 subjects had normal renal function, 349 had mild renal impairment, 82 with moderate renal impairment, and 4 had severe renal impairment. The similarity of the distribution CL across these categories of renal function indicates that renal function does not affect the CL, hence the PK of ipilimumab. Accordingly, ipilimumab can be dosed without regard to renal function.

### Mild Hepatic Impairment has No Effect on Ipilimumab CL.

Some data shows that within the limits of the data analyzed, mild hepatic impairment does not affect ipilimumab clearance. There was only one subject with moderate hepatic impairment in the dataset, and there was none with severe hepatic impairment. The bulk of the PK data came from 708 subjects with normal hepatic function and the remaining 76 subjects had mild hepatic impairment.

### Dacarbazine Did not Affect Ipilimumab CL.

Approximately one third of the subjects in the PPK dataset were on ipilimumab and dacarbazine, while the rest of the subjects were not. It has been shown that the power to detect detection drug-drug interaction using the PPK approach was profoundly affected by intersubject variability, followed by sample size, and the percent of subjects on the combination. The detection of drug-drug in a PPK dataset is possible with one third of the subjects on an interacting drug for an approximate 30% intersubject variability in CL. The estimated intersubject variability in ipilimumab CL was 34.6% in the final PPK model. Thus, the data contained enough information for the detection of the dacarbazine interaction with ipilimumab PK, if present.

Patient Population and Immunogenicity (ADA Status) were not Found to be Clinically Relevant Predictors of Ipilimumab CL.

These covariates and age were not found to be potential clinically relevant predictors of ipilimumab disposition.

### Time Invariance in Ipilimumab PK and Time to Steady State

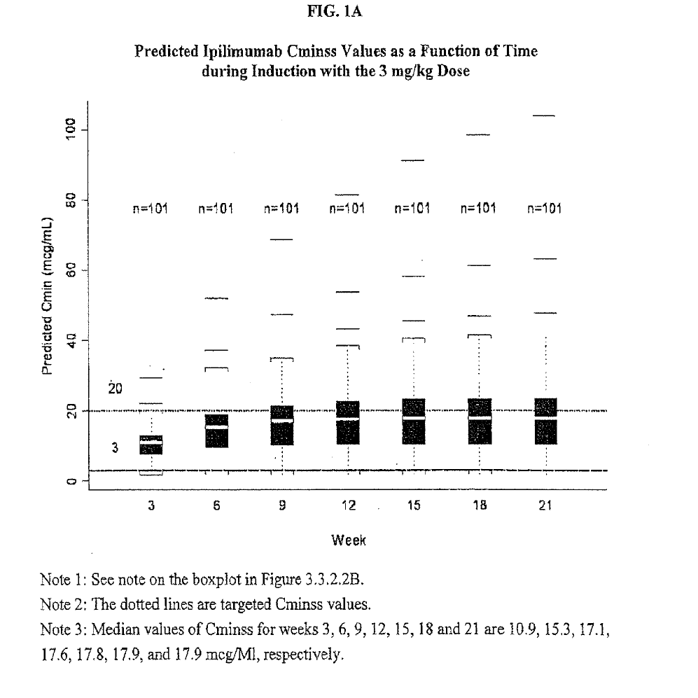

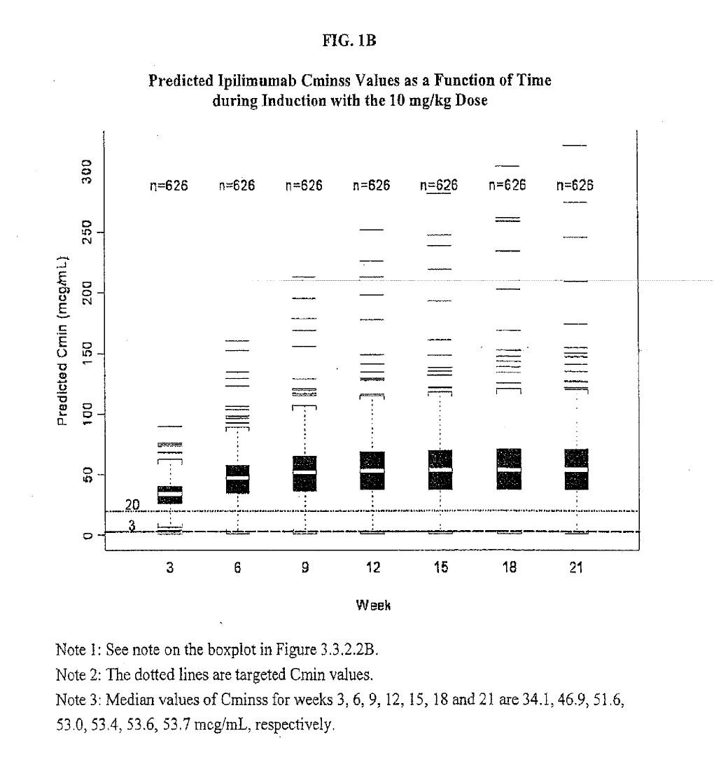

The PK of ipilimumab was shown to be time invariant. Each subject in the final dataset was given seven Q3W doses assuming a nominal 1.5 hour infusion and the Cminss simulated over a 21 week period (see FIG. 1A for the 3 mg/kg dose and FIG. 1B for the 10 mg/kg dose). Ninety percent of the steady-state is reached after the third Q3W dose of ipilimumab. The median Cmin levels off by week nine (i.e., the third dose). In each figure, the reference target trough concentrations are dashed (3 mcg/mL) and dotted (20 mcg/mL) lines.

### Evaluation of Induction Period Dosage Regimen

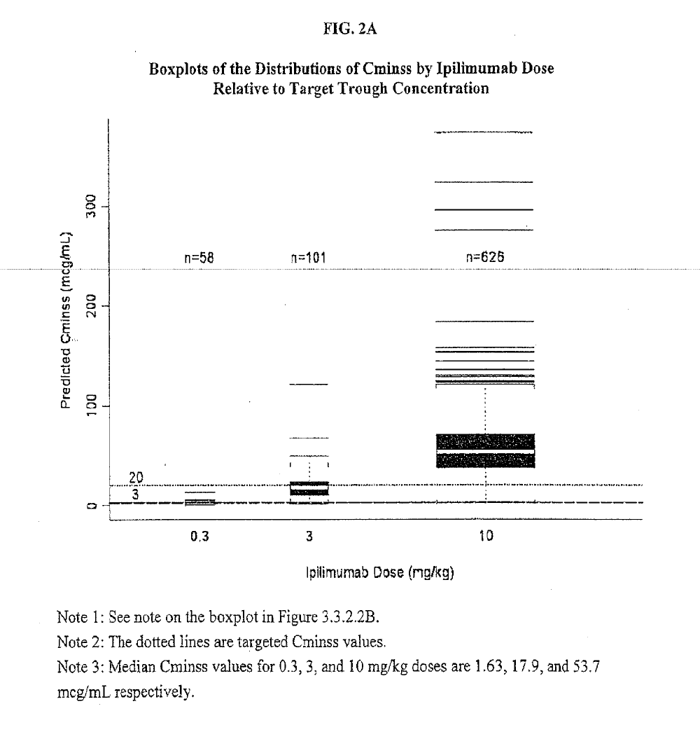

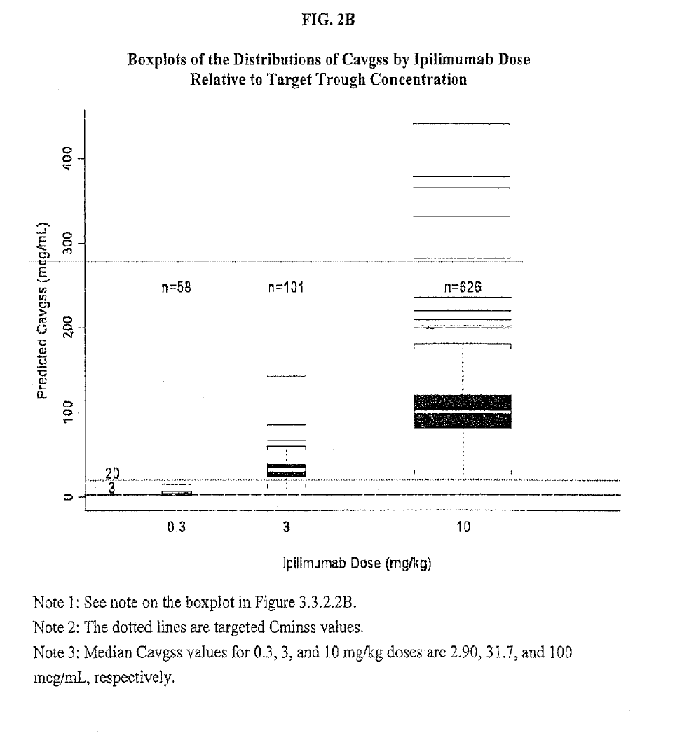

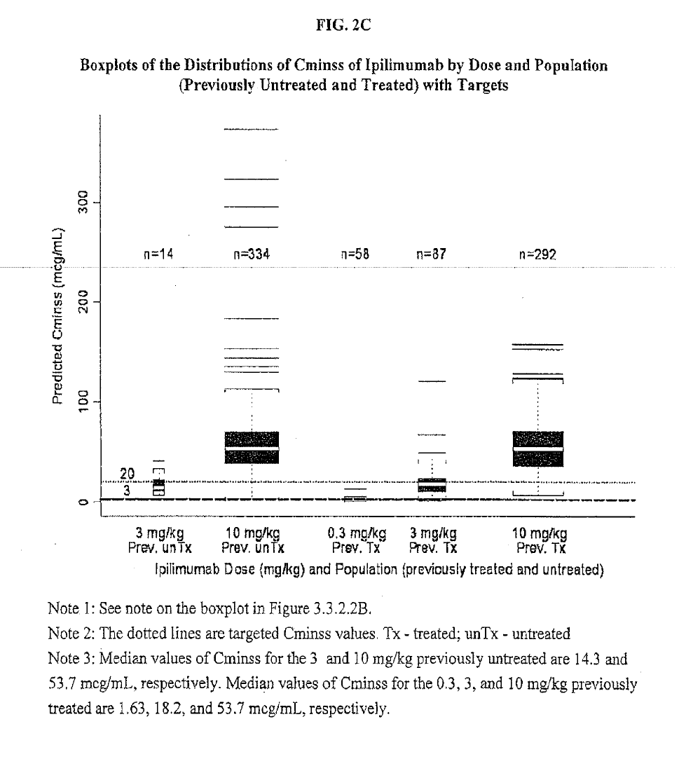

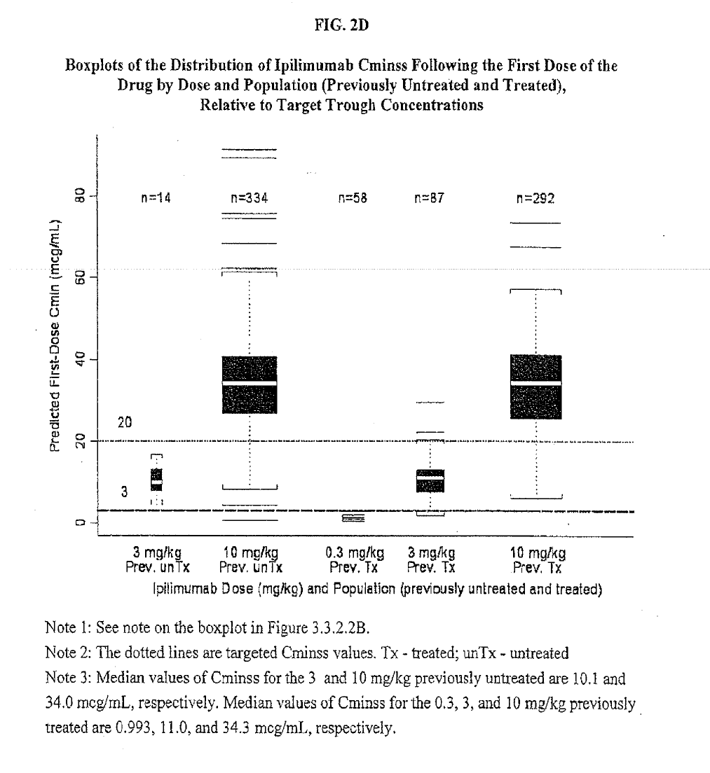

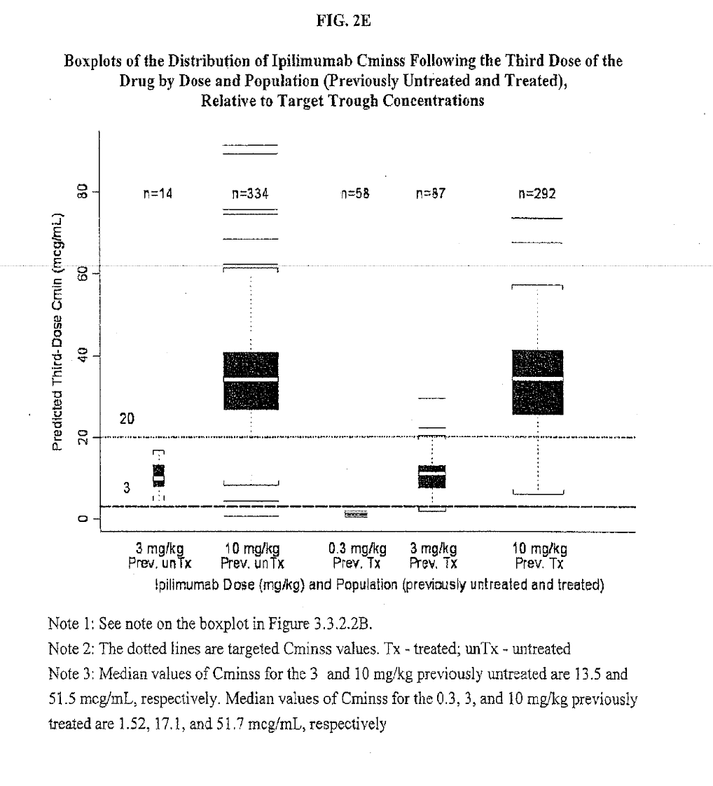

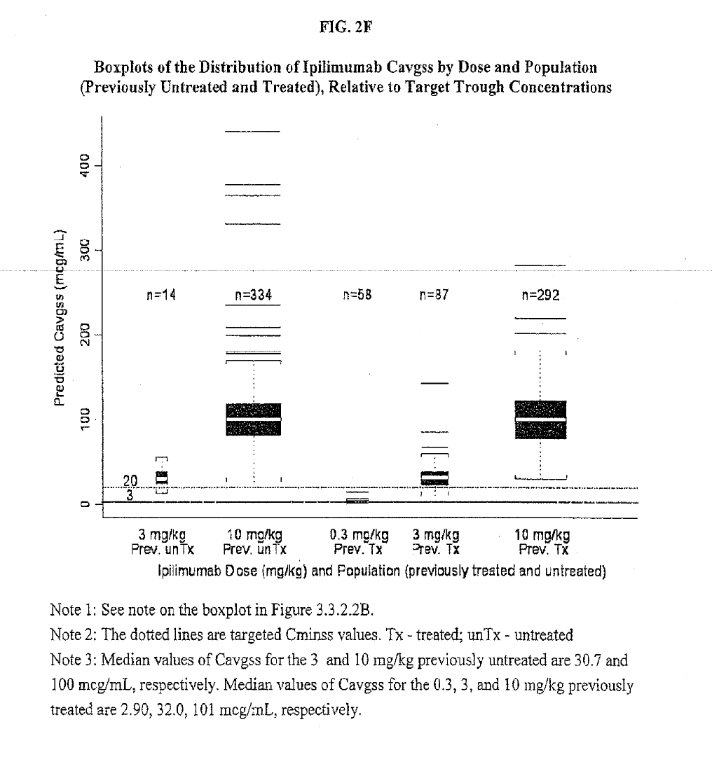

The distribution of Cminss and Cavgss by dose are shown in FIG. 2A and FIG. 2B, respectively. The distribution of model predicted Cminss by dose and population, and by dose and population after the first and third doses are presented in FIG. 2C, FIG. 2D, and FIG. 2E, respectively. FIG. 2F shows the distribution of predicted Cavgss by dose and population. In each figure, the reference target trough concentrations of 3 and 20 mcg/mL are dashed and dotted lines, respectively. The results of the PPK model-based predicted Cminss showed that the ipilimumab target trough concentration of 20 mcg/mL was exceeded by approximately 94.3% of subjects in 10 mg/kg group, and the target trough concentration of 3 mcg/mL was exceeded by approximately 99.8% of subjects in this dose group. Ninety nine percent of the subjects in the 3 mg/kg dose exceeded the 3 mcg/mL ipilimumab target trough concentration. As doses increased from 0.3 to 10 mg/kg in a ratio of 1:10:33 for second line population, the median Cminss increased in a ratio of 1.63: 17.9: 53.7, indicating PK of ipilimumab is linear. A similar trend was observed for the predicted Cminss by population. The Cavgss was similar across population for any given dose, confirming linearity of PK.

Three and 10 Mg/Kg Doses Yield Pharmacologically Active Ipilimumab Steady State Cmin Values when the Target Trough Concentrations of 3 and 20 Mcg/mL are Considered.

Distributions of predicted Cminss showed that the 0.3 mg/kg dose yielded Cminss values below the target trough values. As shown previously, the exposures produced by the 0.3 mg/kg are probably too low. The Cminss values produced by the 0.3 mg/kg dose were below the ipilimumab concentrations needed to inhibit the interaction of CTLA-4 with neither CD80 nor CD86. The 3 mg/kg dose yielded Cminss values that were sufficient for maximal inhibition of CD86, while the 10 mg/kg yielded Cminss values sufficient for maximal inhibition of both CD80 and CD86.

### II. Exposure-Efficacy Response Analysis: OS

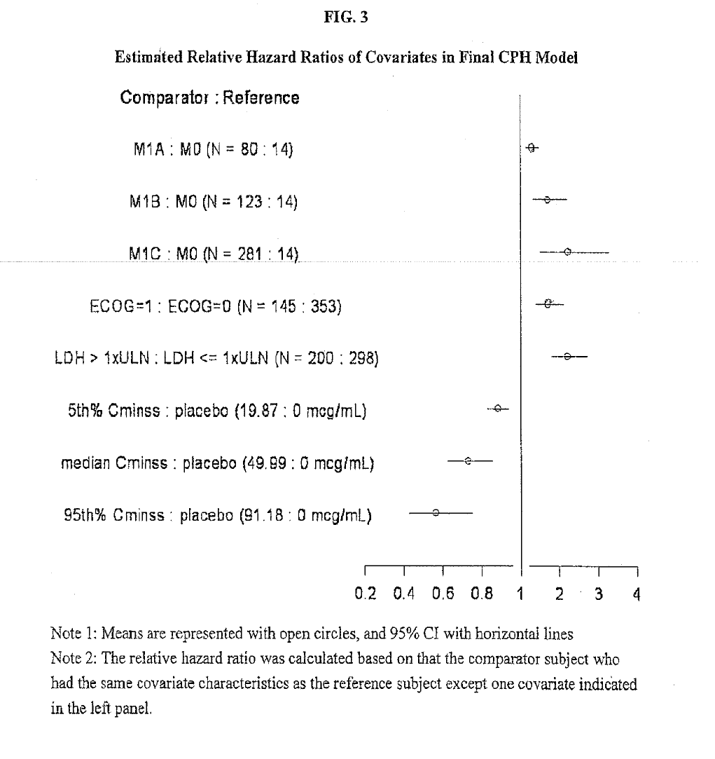

The relationship between the ipilimumab Cminss and OS was characterized with Cox proportional-hazards model (see Table 2 below). The relative hazard ratio results in Table 2 are also presented graphically in FIG. 3. Higher ipilimumab Cminss increases survival. The covariates investigated in the E-R analysis of OS included age, weight, gender, baseline absolute lymphocyte count, baseline lactate dehydrogenase (LDH log-transformed), LDH category (>1×ULN, >2×ULN), ECOG status, and metastatic stage at study entry. Three covariates, LDH status, ECOG status, and metastatic status were identified as significant predictors of OS.

TABLE 2
Parameter Estimates of Final CPH Model for OS Analysis

| | | | Reference | Comparator | Hazard^{a} | Hazard Ratio |
|---|---|---|---|---|---|---|
| predictor | Coefficient β | SE of β | group | group | Ratio | 95% CI |
| Metastatic | 0.268 | 0.066 | M0 | M1A | 1.308 | (1.150, 1.488) |
| status | | | | M1B | 1.711 | (1.322, 2.213) |
| | | | | M1C | 2.237 | (1.521, 3.292) |
| ECOG | 0.538 | 0.110 | ECOG = 0 | ECOG = 1 | 1.712 | (1.381, 2.122) |
| LDH | −0.400 | 0.053 | ELE.LDH | ELE.LDH | 2.224 | (1.808, 2.735) |
| Category^{b} | | | (1) | (−1) | | |
| (>1x ULN) | | | (Normal) | (Elevated) | | |
| Cminss | −0.006 | 0.002 | Cminss = 0 | 5th percentile | 0.884 | (0.831, 0.940) |
| | | | | of Cminss | | |
| | | | | (19.87) | | |
| | | | | median of | 0.733 | (0.627, 0.856) |
| | | | | Cminss | | |
| | | | | (49.99) | | |
| | | | | 95th | 0.567 | (0.427, 0.753) |
| | | | | percentile of | | |
| | | | | Cminss | | |
| | | | | (91.18) | | |

^{a}Hazard ratio represents the hazard ratio for comparator relative to reference predictor variable
^{b}Baseline LDH category

### There was a Significant Relationship Between Ipilimumab Cminss and OS Hazard Ratio.

Cminss was selected as the summary measure of ipilimumab exposure for this analysis based on mechanistic rationale and previous report. The subjects at the 5th percentile of Cminss (19.87 mcg/mL) have an OS hazard ratio of 0.88 relative to the OS of placebo subjects, and subjects at the 95th percentile of Cminss (91.18 mcg/mL) had OS hazard ratio of 0.57 relative to OS of placebo subjects. The relative hazard ratio for a subject who had median Cminss value of 49.9 μg/mL was 0.73 relative to a subject in the placebo (placebo plus dacarbazine) group. In addition, there was a good agreement between the observed and predicted cumulative risk of OS based on Cminss.

Elevated LDH and ECOG Status were Found to be Potentially Clinically Relevant Predictors of OS.

The risk of death in subjects with elevated LDH was 2.22-fold higher than in subjects with normal LDH. The risk of death in subjects with reduced ECOG performance status (ECOG=1) was 1.71-fold than that in subjects with normal ECOG performance status (ECOG=0).

### Metastatic Stage at Study Entry is an Important Predictor of Death.

The more advanced the metastatic stage at study entry, the higher risk of death. The subjects with metastatic stage of M1A, M1B, and M1C have 1.31-, 1.71-, and 2.24-fold higher risk of death, respectively, relative to subjects with metastatic stage M0.

### Although Gender was an Important Predictor of Overall Survival in the Full Model, it was not Retained in the Final Model Based on BIC.

The risk for females was 0.69 that of males in the full model. However, parameter estimates for the other covariates discussed above were quite consistent across all models considered. This was not the case for gender. The coefficient for gender varied greatly from model to model (e.g., >55% difference). Although the dataset may not support the retention of gender as a predictor of OS, a pooled analysis of Eastern Oncology Group trials showed that gender is a prognostic survival variable in metastatic melanoma. Overall, the results of this analysis show that ipilimumab prolongs survival in previously untreated advanced melanoma subjects. Probability of survival increases with increasing ipilimumab Cminss over the range of exposures achieved with the 10 mg/kg dose, and decreases with increasing baseline LDH. Risk of death increases for subjects with reduced performance status compared to those with normal performance status (ECOG=0). Metastatic stage at study entry is also a clinically relevant covariate that affects the subject survival. Metastatic stage at study entry, abnormal LDH, and poor ECOG performance status are known prognostic factors for poorer survival from metastatic melanoma.

III. Exposure-Safety Response Analyses: irAE

The ipilimumab exposure-irAE relationship was well characterized by the Emax-based proportional odds models. Table 3 summarizes the Emax model parameter estimates with 95% confidence intervals for each irAE type. Table 4 summarizes the model covariates for each irAE type.

TABLE 3
Final Exposure-Safety Response Model Estimated Emax Model
Parameter for Each irAE Type

| | | | 95% Confidence |
|---|---|---|---|
| irAE Type | Parameter | Estimate | Interval |
| Any Type | Emax | 3.85 | (3.33, 4.62) |
| | EC50 | 17.8 | (7.87, 33.5) |
| Gastrointestinal | Emax | 2.27 | (1.62, 3.30) |
| | EC50 | 12.9 | (3.09, 42.3) |
| Hepatobiliary | Emax | 5.34 | (4.18, 7.43) |
| | EC50 | 40.8 | (19.6, 84.3) |
| Skin | Emax | 3.87 | (3.05, 6.66) |
| | EC50 | 10.7 | (1.54, 36.7) |

TABLE 4
Final Exposure-Safety Response Model Estimated Covariate Odds Ratios

| | | Comparator: | Odds Ratio | |
|---|---|---|---|---|
| irAE Type | Covariate | Reference | Estimate | 95% Confidence Interval |
| Any | Second Line | Second Line: | 0.510 | (0.337, 0.667) |
| | | First Line | | |
| Gastrointestinal | Dacarbazine | Concomitant | 0.572 | (0.391, 0.853) |
| | | Dacarabazine: | | |
| | | No Dacarbazine | | |
| Liver | Dacarbazine | Concomitant | 9.30 | (6.04, 15.5) |
| | | Dacarabazine: No | | |
| | | Dacarbazine | | |

### EXAMPLE 2

### Quantitative Determination of BMS-734016 (Ipilimumab) in Human Serum by Enzyme-Linked Immunosorbent Assay (ELISA)

### Abstract

BMS-734016 is a fully human IgG1 monoclonal antibody that binds to CTLA-4 antigen expressed on the surface of activated T lymphocytes. An enzyme-linked immunosorbent assay (ELISA) for the quantification of BMS-734016 in human serum was developed and validated. The ELISA method employed a recombinant human CTLA-4/Fc chimera adsorbed onto a microtiter plate to capture BMS-734016 in 0.1% human serum. The captured BMS-734016 was then detected using a commercial purified goat anti-human antibody labeled with alkaline phosphatase. The validation consisted of eight runs for the determination of accuracy, precision and lower limit of quantification (LLOQ), and six runs to determine dilution accuracy. The standard curve, prepared in 100% serum, ranged from 0.400 to 25.6 μg/mL with a quantification range from 0.400 to 25.6 μg/mL and was fitted to a 4-parameter logistic regression model. The inter-assay precision was within 6.82% C.V. and the intra-assay precision was within 5.21% C.V. The assay accuracy was within ±9.40% of their nominal values. Forty-one out of forty-eight of the QC samples at the lower limit of quantification (LLOQ) of 0.400 μg/mL had their calculated concentrations within ±20% of the nominal value. All dilution QC samples at a 1:100-fold dilution had calculated concentrations within ±20% of their nominal values. BMS-734016 is stable at room temperature for twenty-five hours and eight freeze/thaw cycles. Short-term stability of BMS-734016 in human serum was evaluated at −70° C. or below for up to 159 days. The concentration of BMS-734016 can be accurately determined following serial dilution of the sample. Based on the results of this validation, the criteria for sample analysis are as follows: 1) the calculated concentrations of at least three-fourths of all calibration standards shall be within ±20% of their nominal concentrations; 2) at least one replicate of the lowest concentration in the standard curve shall be within ±20% of the nominal concentration for that level to qualify as the LLOQ; 3) the calculated concentrations of at least two-thirds of the analytical quality control (QC) samples shall be within ±20% of their individual nominal concentrations with at least one acceptable QC sample at each level.

### Materials, Solutions, and Reagents

BMS-734016 (Ipilimumab), Bristol-Myers Squibb.
Recombinant human CTLA-4/Fc Chimera, R&D Systems, Catalog #325-CT.
Goat anti-human IgG F(ab)′2 alkaline phosphatase, Jackson ImmunoResearch, Catalog #109-055-097.
Phosphatase Substrate Kit, Pierce, Catalog #37620.
Human serum, Bioreclamation Inc., Hicksville, N.Y.
BupH™ Carbonate-Bicarbonate Buffer Pack, Pierce, Catalog #28382.
Tris Buffered Saline with 1% BSA, pH 8.0, Sigma, Catalog #T-6789.
Tris Buffered Saline with Tween 20, pH 8.0, Sigma, Catalog #T-9039.
2 N Sodium Hydroxide, J. T. Baker, Catalog #5633-02.
Mouse Gamma Globulin, Sigma Catalog #G9894.
Dulbecco's PBS Buffer (10×), cat. #D-1408, Sigma.
Phosphate-buffered saline (PBS): Combined 100 mL of 10×PBS with 900 mL of Milli-Q water.
Coating Buffer, 0.2 M Sodium Carbonate-Bicarbonate Buffer, pH 9.4: One packet of BupH™ Carbonate-Bicarbonate Buffer Pack was dissolved for every 500 mL of Milli-Q water.
Recombinant Human CTLA-4/Fc Chimera Stock, 500 μg/mL: Enough Milli-Q water was added to the vial to prepare a solution of no less than 500 μg/mL.
Assay Buffer, Tris Buffered Saline with 1% BSA: The contents of one packet of TBS with 1% BSA was dissolved in approximately 900 mL Milli-Q water. The final volume was adjusted to 1000 mL with Milli-Q water and filtered through a sterile 0.22 μm filter.
Mouse Gamma Globulin Stock, 10 mg/mL: 50 mg of lyophilized mouse gamma globulin was reconstituted with 5 mL of PBS.
Wash Buffer, Tris Buffered Saline with 0.05% Tween 20: The contents of four Tris buffered saline, 0.05% Tween 20 foil pouches were dissolved in 4 L Milli-Q water.
Goat Anti-human IgG F(ab)′2 Alkaline Phosphatase Stock, 0.6 mg/mL: The reagent was reconstituted with 1.0 mL Milli-Q water.
Stop Solution, 1N NaOH: 250 mL of Milli-Q water were combined with 250 mL of 2N NaOH.
Coating solution, 1.00 μg/mL CTLA-4/Fc Chimera: A 1.00 μg/mL solution was prepared in 0.2 M sodium carbonate-bicarbonate buffer and 100 μL was added per well.
Sample Diluent, 100 μg/mL Mouse Gamma Globulin in Assay Buffer: Mouse Gamma Globulin was diluted to final concentration of 100 μg/mL in Assay Buffer.
Goat Anti-Human IgG Alkaline Phosphatase: A final dilution of 1:2500 was prepared with sample diluent.
pNPP Substrate Working Solution: Two pNPP tablets were dissolved in a solution containing 8 mL of Milli-Q water and 2 mL of Diethanolamine Buffer.

### ELISA Procedure

1) Add 100 μL of the 1.00 μg/mL coating solution to every well of the ELISA plate. Cover with a plate sealer and incubate at 2-8° C. for at least 16 hours.
2) Wash the plate 3 times with 300 μL wash buffer. Rotate plate and repeat wash cycle. Blot plate on paper towel after washing. Wash Program 7.0.1.3.
3) Add 200 μL of assay buffer to each well to block remaining binding sites on the plate. Incubate 1-2 hours at room temperature.
4) Wash the plate 3 times with 300 μL wash buffer. Blot plate on paper towel after washing.
5) Transfer, in duplicate, 100 μL of the diluted standards to the assay plate.
6) Transfer, in triplicate, 100 μL of the diluted QCs to the assay plate.
7) Transfer, in singlicate, 100 μL of the diluted matrix blank and samples to the assay plate.
8) Incubate for 2 hours ±15 minutes at room temperature.
9) Wash the plate 3 times with 300 μL wash buffer. Blot plate on paper towel after washing.
10) Add 100 μL of the alkaline phosphate conjugate (1:2500 in sample diluent) to each well of the assay plate. Incubate for approximately 90 minutes at room temperature.
11) Wash the plate 3 times with 300 μL wash buffer. Blot plate on paper towel after washing.
12) Add 100 μL of substrate to each well of the assay plate and incubate the plates for approximately 30 minutes at room temperature.
13) Add 100 μL of stop solution to each well of the assay plate. Measure OD within 30 minutes with a 405 nm test and a 620 nm reference filter.

### Validation Procedures And Results

### 1. Standard Curve Range

An eight-point calibration standard curve ranging from 0.400 to 25.6 μg/mL of BMS-734016 was assayed in duplicate in each analytical run. Table 5 shows the summary of the individual standard curve data obtained in the eleven runs used to validate the method (Runs 11NMP2 to 21NMP2). In each run, the deviations of the back-calculated concentrations from their nominal values were within ±20% for at least three-fourths of the calibration standards. All of the validation runs passed acceptance criteria. Table 6 shows the standard curve regression analysis results obtained for all eleven runs.

TABLE 5
Individual Standard Curve Concentration Data for BMS-734016 in Human Serum

| | 0.400 | 0.800 | 1.60 | 3.20 | 6.40 | 12.8 | 19.2 | 25.6 |
|---|---|---|---|---|---|---|---|---|
| Run Number | μg/mL | μg/mL | μg/mL | μg/mL | μg/mL | μg/mL | μg/mL | μg/mL |
| 11NMP2 | 0.35 | 0.83 | 1.65 | 3.20 | 6.36 | 13.04 | 19.65 | 26.31 |
| 11NMP2 | 0.37 | 0.80 | 1.62 | 3.23 | 6.30 | 12.59 | 19.00 | 24.69 |
| 12NMP2 | 0.42 | 0.77 | 1.64 | 3.21 | 6.57 | 13.20 | 19.84 | 26.52 |
| 12NMP2 | 0.40 | 0.78 | 1.62 | 3.15 | 6.26 | 12.37 | 18.63 | 24.69 |
| 13NMP2 | A | 0.80 | 1.59 | 3.31 | 6.51 | 13.07 | 19.36 | 26.27 |
| 13NMP2 | 0.45 | 0.77 | 1.52 | 3.18 | 6.25 | 12.67 | 18.75 | 25.12 |
| 14NMP2 | 0.42 | 0.79 | 1.62 | 3.32 | 6.56 | 13.04 | 19.81 | 26.28 |
| 14NMP2 | 0.43 | 0.75 | 1.54 | 3.17 | 6.22 | 12.48 | 18.74 | 24.87 |
| 15NMP2 | 0.37 | 0.79 | 1.62 | 3.24 | 6.46 | 12.96 | 19.57 | 26.61 |
| 15NMP2 | 0.43 | 0.79 | 1.60 | 3.20 | 6.26 | 12.70 | 18.86 | 24.59 |
| 16NMP2 | 0.39 | 0.80 | 1.67 | 3.25 | 6.48 | 12.86 | 19.94 | 25.95 |
| 16NMP2 | 0.37 | 0.79 | 1.63 | 3.15 | 6.28 | 12.59 | 18.92 | 24.96 |
| 17NMP2 | 0.35 | 0.92 | 1.62 | 3.25 | 6.38 | 12.15 | 20.25 | 25.24 |
| 17NMP2 | 0.32 | 0.78 | 1.62 | 3.19 | 6.37 | 12.95 | 19.46 | 25.14 |
| 18NMP2 | 0.38 | 0.82 | 1.67 | 3.26 | 6.54 | 12.92 | 20.03 | 25.94 |
| 18NMP2 | 0.35 | 0.80 | 1.61 | 3.13 | 6.24 | 12.44 | 19.04 | 24.83 |
| 19NMP2 | A | A | A | 3.46 | 6.42 | 12.97 | 19.36 | 25.95 |
| 19NMP2 | A | 0.67 | 1.81 | 2.94 | 6.21 | 12.67 | 19.30 | 25.04 |
| 20NMP2 | 0.38 | 0.82 | 1.62 | 3.24 | 6.55 | 12.99 | 19.84 | 25.68 |
| 20NMP2 | 0.39 | 0.79 | 1.61 | 3.15 | 6.30 | 12.28 | 19.18 | 25.17 |
| 21NMP2 | 0.46 | 0.75 | 1.55 | 3.34 | 6.71 | 10.77 | 19.65 | 25.40 |
| 21NMP2 | 0.45 | 0.73 | 1.50 | 3.15 | 6.59 | 13.84 | 19.79 | 25.46 |
| Mean | 0.39 | 0.79 | 1.62 | 3.21 | 6.40 | 12.71 | 19.41 | 25.49 |
| SD | 0.04 | 0.05 | 0.06 | 0.10 | 0.14 | 0.57 | 0.47 | 0.64 |
| % CV | 9.75 | 5.83 | 3.94 | 3.10 | 2.26 | 4.45 | 2.41 | 2.52 |
| % Deviation | −1.67 | −1.60 | 1.04 | 0.44 | 0.01 | −0.73 | 1.09 | −0.44 |
| n | 19 | 21 | 21 | 22 | 22 | 22 | 22 | 22 |

Legend A Deleted from calculations due to unacceptable quantification per BMS SOPs.

TABLE 6
Standard Curve Regression Analysis Results for BMS-734016 in Human Serum

| | Curve | | | | | | LLOQ, | ULOQ, |
|---|---|---|---|---|---|---|---|---|
| Run Date | Number | a | b | c | d | R | μg/mL | μg/mL |
| 16 Dec. 2005 | 11NMP2 | 0.0750 | 1.08 | 20.1 | 2.39 | 0.9998 | 0.400 | 25.6 |
| 16 Dec. 2005 | 12NMP2 | 0.0687 | 1.04 | 24.8 | 2.87 | 0.9996 | 0.400 | 25.6 |
| 19 Dec. 2005 | 13NMP2 | 0.0543 | 0.971 | 26.3 | 2.93 | 0.9998 | 0.400 | 25.6 |
| 19 Dec. 2005 | 14NMP2 | 0.0639 | 1.01 | 23.8 | 2.88 | 0.9997 | 0.400 | 25.6 |
| 20 Dec. 2005 | 15NMP2 | 0.0766 | 1.07 | 18.2 | 2.73 | 0.9998 | 0.400 | 25.6 |
| 20 Dec. 2005 | 16NMP2 | 0.0804 | 1.06 | 21.0 | 3.03 | 0.9998 | 0.400 | 25.6 |
| 21 Dec. 2005 | 17NMP2 | 0.0659 | 1.07 | 19.3 | 2.04 | 0.9996 | 0.400 | 25.6 |
| 21 Dec. 2005 | 18NMP2 | 0.0699 | 1.15 | 17.2 | 2.00 | 0.9998 | 0.400 | 25.6 |
| 29 Dec. 2005 | 19NMP2 | 0.0985 | 1.03 | 23.5 | 2.62 | 0.9996 | 0.800 | 25.6 |
| 29 Dec. 2005 | 20NMP2 | 0.0826 | 1.12 | 21.2 | 2.63 | 0.9998 | 0.400 | 25.6 |
| 5 Jan. 2006 | 21NMP2 | 0.0250 | 0.776 | 69.0 | 4.48 | 0.9982 | 0.400 | 25.6 |
| Mean | | 0.0692 | 1.03 | 25.9 | 2.78 | 0.9996 | | |
| S.D. | | 0.0186 | 0.0983 | 14.6 | 0.659 | 0.000473 | | |
| % CV | | 26.9 | 9.51 | 56.3 | 23.7 | 0.0473 | | |
| n | | 11 | 11 | 11 | 11 | 11 | | |

The standard curve is fitted using the following four-parameter logistic model:
$Y = \frac{A - D}{1 + ( X / C )} + D$ where:
X = Concentration
Y = Response
A = “0” dose response
B = slope factor
C = ED₅₀
D = Infinite dose (NSB) response

### 2. Accuracy and Precision

The accuracy and precision of the method were assessed by analyzing QC samples at concentrations within the lower, middle and upper quartiles of the standard curve as well as an LLOQ QC. A sixth QC sample with a concentration higher than the upper limit of the standard curve range was also analyzed. This QC sample, known as the dilution QC, was serially diluted with the other QC samples as described in section 3.3.2 then diluted 1:100. Six replicates at each concentration (0.400, 1.20, 3.90, 12.8 and 20.5 μg/mL) were analyzed together with two replicates of each standard in eight separate accuracy and precision runs (11NMP2 to 18NMP2). Six replicates at the dilution concentration (500 μg/mL) were analyzed together with two replicates of each standard in six separate accuracy and precision runs (11NMP2 to 16NMP2). The accuracy of the method was assessed by calculating the deviation of the calculated concentrations from their nominal values. The intra- and inter-assay precision data were assessed by calculating the CV values.

The individual QC data obtained in the eleven runs were used to validate the method. In each run the deviations of the calculated concentrations from their nominal values were within ±20% for at least two-thirds of the QC samples. The results of one-way ANOVA for the eight runs (six runs for the dilution QC) used to determine the accuracy and precision of the method are shown in Table 7. The intra-assay precision data were within 5.21% CV and inter-assay precision data were within 6.82% CV. The assay accuracy data were within ±9.40% of the nominal values. At the lower limit of quantification of 0.400 μg/mL, the intra-assay precision was 9.37% CV and interassay precision was 10.52% CV. The assay accuracy was within ±9.88% of the nominal value. For the dilution QC at 500 μg/mL, the intra-assay precision was 4.47% CV and inter-assay precision was 3.92% CV. The assay accuracy was within ±9.52% of the nominal value.

TABLE 7
Accuracy and Precision for BMS-734016 in Human Serum

| | Nominal Conc. | | | | | |
|---|---|---|---|---|---|---|
| | 1.2 | 3.9 | 12.8 | 20.5 | 0.400 | 500 |
| | μg/mL | μg/mL | μg/mL | μg/mL | μg/mL | μg/mL |
| Mean Observed | 1.31 | 4.24 | 13.76 | 21.38 | 0.36 | 452.42 |
| Conc., μg/mL | | | | | | |
| % Dev. | 9.40 | 8.80 | 7.51 | 4.28 | −9.88 | −9.52 |
| Inter-assay | 6.82 | 4.51 | 4.71 | 4.95 | 10.52 | 3.92 |
| Precision | | | | | | |
| (% CV) | | | | | | |
| Intra-assay | 4.78 | 5.21 | 3.73 | 3.57 | 9.37 | 4.47 |
| Precision | | | | | | |
| (% CV) | | | | | | |
| n | 48 | 48 | 48 | 48 | 48 | 36 |
| Number of | 8 | 8 | 8 | 8 | 8 | 6 |
| Runs | | | | | | |

### 3. Lower Limit of Quantification

The lower limit of quantification (LLOQ) of BMS-734016 was assessed using 10 individual serum samples spiked at 0.400 μg/mL, the lowest concentration in the quantification range. The LLOQ samples were processed and analyzed with a standard curve and QC samples, and their calculated concentrations determined (Run 21NMP2). The results of the LLOQ determinations at 0.400 μg/mL are shown in Table 8. The deviations of the calculated concentrations from the nominal value were within ±20% for all of the LLOQ samples. These results support the use of 0.400 μg/mL in 100% serum as the LLOQ.

TABLE 8
Lower Limit of Quantification Determination of BMS-734016 in
Human Serum (μg/mL) Run 21NMP2

| Individual | Nominal Conc. | Calculated Conc. | | Mean | Mean |
|---|---|---|---|---|---|
| Sera | μg/mL | μg/mL | % Dev. | Conc. | % Dev. |
| 1 | 0.400 | 0.43 | 7.43 | 0.40 | 0.29 |
| | 0.400 | 0.42 | 4.12 | | |
| | 0.400 | 0.42 | 4.12 | | |
| 2 | 0.400 | 0.40 | −0.80 | | |
| | 0.400 | 0.39 | −2.42 | | |
| | 0.400 | 0.40 | −0.80 | | |
| 3 | 0.400 | 0.36 | −10.44 | | |
| | 0.400 | 0.37 | −7.26 | | |
| | 0.400 | 0.36 | −8.85 | | |
| 4 | 0.400 | 0.38 | −4.04 | | |
| | 0.400 | 0.39 | −2.42 | | |
| | 0.400 | 0.38 | −4.04 | | |
| 5 | 0.400 | 0.44 | 9.09 | | |
| | 0.400 | 0.43 | 7.43 | | |
| | 0.400 | 0.44 | 10.76 | | |
| 6 | 0.400 | 0.40 | −0.80 | | |
| | 0.400 | 0.39 | −2.42 | | |
| | 0.400 | 0.39 | −2.42 | | |
| 7 | 0.400 | 0.42 | 4.12 | | |
| | 0.400 | 0.42 | 5.77 | | |
| | 0.400 | 0.42 | 5.77 | | |
| 8 | 0.400 | 0.35 | −12.03 | | |
| | 0.400 | 0.36 | −10.44 | | |
| | 0.400 | 0.36 | −10.44 | | |
| 9 | 0.400 | 0.41 | 2.47 | | |
| | 0.400 | 0.41 | 2.47 | | |
| | 0.400 | 0.41 | 2.47 | | |
| 10 | 0.400 | 0.44 | 9.09 | | |
| | 0.400 | 0.43 | 7.43 | | |
| | 0.400 | 0.42 | 5.77 | | |

### 4. Stability

### 1) Bench Top Stability of BMS-734016 in Human Serum

The bench top stability of BMS-734016 in human serum at room temperature was evaluated using QC samples (n=6) spiked at 1.20, 20.5 and 500 μg/mL. The test samples were allowed to remain at room temperature for 25 hours prior to analysis. The deviations of the mean calculated concentrations of the test QC samples from the nominal concentrations were used as an indicator of the room temperature stability of BMS-734016 in human serum. BMS-734016 was stable for up to 25 hours at room temperature.

### 2) Freeze/Thaw Stability of BMS-734016 in Human Serum

The freeze-thaw stability of BMS-734016 in human serum was assessed after three and eight freeze-thaw cycles using QC samples (n=6) spiked at 1.20, 20.0 and 500 μg/mL. These QC samples were frozen at −70° C. or below and thawed at room temperature in a manner consistent with typical sample analysis. Samples were processed and analyzed after three and eight thawing cycles to determine the BMS-734016 concentrations. The deviations of the mean calculated concentrations of the test QC samples from the nominal concentration were used as an indicator of the freeze/thaw stability of BMS-734016 in human serum. BMS-734016 was stable for up to eight freeze/thaw cycles.

### 3) Short Term Matrix Stability of BMS-734016 in Human Serum

Short term frozen matrix stability of BMS-734016 in human serum at −70° C. or below was evaluated (n=6) using QC samples at 1.20, 20.0 and 500 μg/mL stored for a period of 183 days (1.20 and 20.0 μg/mL) and 159 days (500 μg/mL). The deviations of the mean calculated concentrations of the QC samples from the nominal concentrations were used as an indicator of the −70° C. or below stability of BMS-734016 in human serum. BMS-734016 was stable for up to 183 days (1.20 and 20.0 μg/mL) and 159 days (500 μg/mL) at −70° C. or below.

### 5. Dilution Linearity of Serum Samples

To demonstrate dilutional linearity of serum samples, the 500 μg/mL dilution quality control prepared in human serum was serially diluted in assay buffer at different dilutions such that the resulting OD units would fall in all four quartiles of the standard curve range prior to the MRD selected. The results indicated that the calculated concentrations of the individually diluted test samples were within ±20.0% of the nominal value for all samples diluted to concentrations within the quantification range.

### 6. Prozone or “Hook Effect”

The prozone or “hook effect” may cause the inhibition of assay response due to excess analyte concentrations. The prozone was evaluated by analyzing the dilution QC at a concentration of 500 μg/mL. This high concentration pool was analyzed at four additional serial dilutions in addition to the minimum required dilution. The results of this experiment demonstrated that the “hook effect” will not present a problem up to sample concentrations of 500 μg/mL.

### EQUIVALENTS

Those skilled in the art will recognize or be able to ascertain, using no more than routine experimentation, many equivalents of the specific embodiments of the invention described herein. Such equivalents are intended to be encompassed by the following claims.

1. A method of treating a cancer in a subject in need of treatment, comprising:
(a) administering to the subject a predetermined dosage of an anti-CTLA4 antibody;
(b) detecting the level of the anti-CTLA4 antibody in a sample of the subject; and
(c) increasing the dosage of the anti-CTLA4 antibody in the subject if the level of the anti-CTLA4 antibody from step (b) is below a threshold exposure level, such that the cancer is treated in the subject.

2. The method of claim 1, wherein the anti-CTLA4 antibody is a human antibody.

3. The method of claim 2, wherein the anti-CTLA4 antibody is MDX-010.

4. The method of claim 1, wherein the anti-CTLA4 antibody comprises:
(a) a heavy chain variable region comprising the amino acid sequence of SEQ ID NO: 1; and
(b) a light chain variable region comprising the amino acid sequence of SEQ ID NO: 2.

5. The method of claim 4, wherein the anti-CTLA4 antibody comprises:
(a) a heavy chain variable region CDR1 comprising SEQ ID NO: 3;
(b) a heavy chain variable region CDR2 comprising SEQ ID NO: 4;
(c) a heavy chain variable region CDR3 comprising SEQ ID NO: 5;
(d) a light chain variable region CDR1 comprising SEQ ID NO: 6;
(e) a light chain variable region CDR2 comprising SEQ ID NO: 7; and
(f) a light chain variable region CDR3 comprising SEQ ID NO: 8.

6. The method of claim 1, wherein the level of the anti-CTLA4 antibody is detected by an immunoassay in step (b).

7. The method of claim 6, wherein the immunoassay comprises contacting said sample with an antigen which binds to the anti-CTLA4 antibody under conditions suitable for antibody-antigen complex formation, followed by the detection of the antibody-antigen complex formation.

8. The method of claim 7, wherein the antigen is a CTLA4 protein.

9. The method of claim 7, wherein said detection is accomplished by a means selected from the group consisting of EIA, ELISA, RIA, indirect competitive immunoassay, direct competitive immunoassay, non-competitive immunoassay, sandwich immunoassay, and agglutination assay.

10. The method of claim 1, wherein the cancer is selected from the group consisting of melanoma, prostate cancer, lung cancer, gastric cancer, ovarian cancer, breast cancer, and glioblastoma.

11. The method of claim 10, wherein the cancer is melanoma.

12. The method of claim 1, wherein the predetermined dosage is 3 mg/kg or 10 mg/kg of body weight.

13. The method of claim 1, wherein the subject is further administered with another anti-cancer agent.

14. The method of claim 1, wherein the subject was previously treated for the cancer.

15. The method of claim 1, wherein the subject was previously not treated for the cancer.

16. A method of decreasing clearance of a therapeutic anti-CTLA4 antibody in a subject in need of treatment of a cancer, comprising:
(a) administering to the subject a predetermined dosage of an anti-CTLA4 antibody;
(b) detecting the level of the anti-CTLA4 antibody in a sample of the subject; and
(c) increasing the dosage of the anti-CTLA4 antibody in the subject if the level of the anti-CTLA4 antibody from step (b) is below a threshold exposure level, such that clearance of the anti-CTLA4 antibody is decreased in the subject.

17. The method of claim 16, wherein the cancer is melanoma.

18. A kit comprising: (1) an antigen which specifically binds to an anti-CTLA4 antibody; and (2) reagents necessary for facilitating an antibody-antigen complex formation.

19. The kit of claim 18, further comprising the anti-CTLA4 antibody as a control.

20. The kit of claim 19, wherein the anti-CTLA4 antibody is MDX-010.
